SEQUENCE LISTING (17 sequences)

SEQ ID NO: 1 - protein, 346 aa, Mycobacterium tuberculosis
MDFTIFPPEFNSLNIQGSARPFLVAANAWKNLSNELSYAASRFESEINGLITSWRGPSSTIMAAAVAPFRAWIVTTASLAELVADHISVVAGAYEAAHAAHVPLPVIETNRLTRLALATTNIFGIHTPAIFALDALYAQYWSQDGEAMNLYATMAAAAARLTPFSPPAPIANPGALARLYELIGSVSETVGSFAAPATKNLPSKLWTLLTKGTYPLTAARISSIPVEYVLAFVEGSNMGQMMGNLAMRSLTPTLKGPLELLPNAVRPAVSATLGNADTIGGLSVPPSWVADKSITPLAKAVPTSAPGGPSGTSWAQLGLASLAGGAVGAVAARTRSGVILRSPAAG

SEQ ID NO: 2 - DNA, 1041 bp, Mycobacterium tuberculosis
atggatttcacaatttttccgccggagttcaactccctcaacatccaaggtagcgctcgtccgtttctagtagccgcgaacgcctggaagaatctgtccaacgagctgagctacgcggccagtcggttcgagagtgagatcaacgggctgatcacatcgtggcgggggccatcgtcgacgatcatggcagctgcggtcgccccatttcgggcctggattgtcacgaccgcttccctggctgaactcgtcgccgaccacatcagcgtcgtggcaggcgcctatgaagcggcgcacgcagcacacgtgccgctgccggtgatcgagaccaaccgactgacgcgcctcgctctcgccacgaccaacattttcgggattcacacccccgcgatctttgccctcgatgcactgtatgcccagtactggtcccaagatggcgaggcgatgaacctctacgccacaatggcggcggccgccgcacggctgacaccgttctcgcccccggcgccgatcgccaacccgggcgcgctggccagactttatgaactgatcggttcggtgtccgagacggtggggtcattcgccgcgccggcgaccaagaatctgccttcgaagctgtggacgctgttgacgaagggcacctacccgctcacagccgcgcgaatctcgtcgatacccgtggaatacgtgttggcctttgtcgagggcagcaacatgggccagatgatgggcaacctcgccatgcggagcctgacacccacgctcaagggcccgctggagttgctacccaacgcggtcaggcccgcggtgtcggcaacattgggaaatgcggatacgatcggggggttgtcggtgccccccagctgggttgcggacaaatcgattacgccgttggccaaagccgtcccgacctccgcgccgggcggtccgtcgggaacctcgtgggcccagctgggattggcaagcctggccgggggcgctgtgggcgccgtcgcggcaagaacccgttccggagtgatactgcggtcacccgccgccggctag

SEQ ID NO: 3 - DNA, 26 bp, Artificial Sequence
  Description of Artificial Sequence Synthetic primer
gactcgagatggatttcacaattttt

SEQ ID NO: 4 - DNA, 33 bp, Artificial Sequence
  Description of Artificial Sequence Synthetic primer
gcaagcttctagccggcggcgggtgaccgcagt

SEQ ID NO: 5 - protein, 9 aa, Mycobacterium tuberculosis
FNSLNIQGS

SEQ ID NO: 6 - protein, 7 aa, Mycobacterium tuberculosis
WKNLSNE

SEQ ID NO: 7 - protein, 10 aa, Mycobacterium tuberculosis
SYAASRFESE

SEQ ID NO: 8 - protein, 8 aa, Mycobacterium tuberculosis
TSWRGPSS

SEQ ID NO: 9 - protein, 6 aa, Mycobacterium tuberculosis
TNRLTR

SEQ ID NO: 10 - protein, 12 aa, Mycobacterium tuberculosis
AQYWSQDGEAMN

SEQ ID NO: 11 - protein, 8 aa, Mycobacterium tuberculosis
PATKNLPS

SEQ ID NO: 12 - protein, 4 aa, Mycobacterium tuberculosis
TYPL

SEQ ID NO: 13 - protein, 6 aa, Mycobacterium tuberculosis
SNMGQM

SEQ ID NO: 14 - protein, 6 aa, Mycobacterium tuberculosis
TPTLKG

SEQ ID NO: 15 - protein, 5 aa, Mycobacterium tuberculosis
WVADK

SEQ ID NO: 16 - protein, 10 aa, Mycobacterium tuberculosis
APGGPSGTSW

SEQ ID NO: 17 - protein, 9 aa, Mycobacterium tuberculosis
  MOD_RES (2)..(3): Any amino acid
GXXSVPXXW

(19) United States
(12) Patent Application Publication
(10) Pub. No.: US 2010/0159485 A1
(43) Pub. Date: Jun. 24, 2010

(54) DETECTION OF MYCOBACTERIUM TUBERCULOSIS

(75) Inventors: Sangita Mukhopadhyay, Hyderabad (IN); Nooruddin Khan, New Delhi (IN)

Correspondence Address: FOLEY & LARDNER LLP, 111 HUNTINGTON AVENUE, 26TH FLOOR, BOSTON, MA, 02199-7610, US

(73) Assignee: Centre for DNA Fingerprinting and Diagnostics

(21) Appl. No.: 12/339,524

(22) Filed: Dec. 19, 2008

(51) Int. Cl. G01N 33/569; C12Q 1/04

(52) U.S. Cl. 435/7.32; 435/34

(57) ABSTRACT

The present technology relates generally to the use of an Rv1168c polypeptide, or fragments thereof, or Rv1168c polypeptide binding agent for detecting or diagnosing exposure to Mycobacterium tuberculosis as well as detecting or diagnosing Mycobacterium tuberculosis infection in a mammal.

### BACKGROUND

Tuberculosis (TB) remains a significant global public health concern and is a major cause of death in adults by a single bacterial agent (39). The diagnosis of a majority of the TB cases in developing countries like India rely on acid-fast staining of sputum or positive cultures of M. tuberculosis (Mtb) in conjunction with an assessment of clinical symptoms and radiographic information (6, 29, 38). However, these techniques are usually expensive, tedious and time consuming. The most common method employed for detection of Mtb infection is the purified protein derivative (PPD) or tuberculin skin test, but PPD is a crude and poorly defined mixture of mycobacterial antigens, many of which are shared with proteins from the vaccine strain Mycobacterium bovis Bacille Calmette-Guérin (BCG) and from non-tuberculosis environmental mycobacteria (NTM) (20, 22). Therefore, the clinical relevance of a tuberculin test with PPD is not highly reliable (17, 30).

### SUMMARY

The present technology relates generally to the use of an Rv1168c polypeptide or Rv1168c polypeptide binding agent for detecting or diagnosing exposure to Mycobacterium tuberculosis, as well as detecting or diagnosing Mycobacterium tuberculosis infection in a mammal.

In one aspect, the methods are directed to detecting exposure of a mammal to Mycobacterium tuberculosis or diagnosing Mycobacterium tuberculosis infection in a mammal. Such methods include incubating a test biological sample from a mammal with an Rv1168c polypeptide comprising the amino acid sequence of SEQ ID NO: 1, or a fragment thereof, under conditions suitable for an antibody, present in the test biological sample, to bind the Rv1168c polypeptide, or fragment thereof, to form an Rv1168c polypeptide-antibody complex; measuring the level of the Rv1168c polypeptide-antibody complex in the test biological sample; and comparing the level of the Rv1168c polypeptide-antibody complex in the test biological sample to the level of Rv1168c polypeptide-antibody complex detected in a control sample, wherein a greater level of the Rv1168c polypeptide-antibody complex in the test biological sample, compared to the level of the Rv1168c polypeptide-antibody complex in the control sample, is indicative of exposure of the mammal to Mycobacterium tuberculosis and/or Mycobacterium tuberculosis infection in the mammal.

In additional embodiments, the methods are directed to detecting exposure of a mammal to Mycobacterium tuberculosis or diagnosing Mycobacterium tuberculosis infection in a mammal. Such methods include incubating a test biological sample from a mammal with an Rv1168c polypeptide binding agent under conditions suitable for the Rv1168c polypeptide binding agent to bind an Rv1168c polypeptide, or a fragment thereof present in the test biological sample, and form an Rv1168c binding agent-Rv1168c polypeptide complex; and determining the presence or absence of the Rv1168c polypeptide binding agent-Rv1168c polypeptide complex in the test biological sample wherein the presence of Rv1168c polypeptide binding agent-Rv1168c polypeptide complex in the test biological sample is indicative of exposure of the mammal to Mycobacterium tuberculosis and/or a Mycobacterium tuberculosis infection in the mammal.

In other embodiments, the methods of the present technology relate to methods for detecting active Mycobacterium tuberculosis infection in a mammal comprising contacting a test population of peripheral blood mononuclear cells (PBMCs) from the mammal with an Rv1168c polypeptide comprising the amino acid sequence of SEQ ID NO: 1, or a fragment thereof; measuring the level of an at least one cytokine expressed by the test population of PBMCs; and comparing the level of the at least one cytokine expressed by the test population of PBMCs to the level of the at least cytokine measured in a reference population of PBMCs, wherein the expression of a greater or lesser level of the at least one cytokine in the test population of PBMCs compared to the level of the at least one cytokine in the reference population of PBMCs is indicative of active Mycobacterium tuberculosis infection in the mammal.

In certain embodiments the level of the at least one cytokine expressed by the test population of PBMCs is about two times greater than the level of the at least one cytokine measured in the reference population of PBMCs. The at least one cytokine measured in the methods of the present technology includes, but is not limited to, an interleukin type cytokine (e.g., IL-2 subfamily; the IL-10 subfamily; interleukin-5 (IL-5) and the interferon (IFN) subfamily (e.g., γ-interferon)). Additional embodiments of the present technology also include methods wherein the test population of PBMCs are incubated between about 1 day and about 6 days prior to measuring the level of the at least one cytokine expressed by the test population of PBMCs.

In certain embodiments, the mammal displays symptoms of Mycobacterium tuberculosis infection. In other embodiments, the Mycobacterium tuberculosis infection is an extrapulmonary Mycobacterium tuberculosis infection or the mammal's sputum is smear-negative for acid-fast bacilli.

In additional embodiments the Rv1168c polypeptide used in the methods of the present technology comprises, consists essentially of, or consists of the amino acid sequence of SEQ ID NO:1, or fragments thereof. In other embodiments, the Rv1168c polypeptide is fused to a heterologous polypeptide such as a purification facilitating polypeptide (e.g., a polyhistidine tag).

In other embodiments, the test biological sample used in the methods of the present technology includes, but is not limited to, any biological fluid from the mammal such as, but not limited to, whole blood, sputum, blood serum, plasma, saliva, cerebrospinal fluid and urine.

In additional embodiments, the control sample used in the methods of the present technology includes, but is not limited to, e.g., a biological sample from a mammal which has not been exposed to Mycobacterium tuberculosis (e.g., an individual that is uninfected or non-reactive to Mycobacterium tuberculosis), a mammal which has been vaccinated with a tuberculosis vaccine such as Bacille Calmette-Guérin, or a historical biological sample (e.g., a sample taken from a mammal at an earlier time point or a historical data set from patients tested at an earlier time point). The control sample may be a biological sample from the same or different individual or from the same or different species or genus. One of ordinary skill in the art would understand the term “exposed” to include, but is not limited to, the condition of being subjected to something, such as an infectious agent, which may or may not result in an infection and/or a detectable immune response.

In other embodiments, the level of Rv1168c polypeptide-antibody complex or Rv1168c binding agent-Rv1168c polypeptide complex in the test biological sample and control sample is measured or detected by an assay. Additionally, the level of at least one cytokine expressed by the test population of PBMCs and the reference population of PBMCs is measured or detected by an assay. Assays for use in the methods of the present technology include, but are not limited to, e.g., an enzyme immunoassay; an enzyme-linked immunosorbent assay a radioimmunoassay; a rapid flow through assay; or a competitive assay.

In certain embodiments, the level of Rv1168c polypeptide-antibody complex or Rv1168c polypeptide binding agent-Rv1168c polypeptide complex in the control sample is a historical level from a reference sample (e.g., the level of Rv1168c polypeptide-antibody complex or polypeptide binding agent-Rv1168c polypeptide complex in a biological sample taken previously and/or measured previously from the same or different individual or individuals).

In another aspect, kits are provided to assay for an anti-Rv1168c polypeptide antibody or a Rv1168c polypeptide, or fragment thereof, in a biological sample. In certain embodiments, the kits comprise an Rv1168c polypeptide, or fragment thereof, and/or an anti-Rv1168c antibody, or fragment thereof, and instructions for their use.

The foregoing summary is illustrative only and is not intended to be in any way limiting. In addition to the illustrative aspects, embodiments, and features described above, further aspects, embodiments, and features will become apparent by reference to the drawings and the following detailed description.

### BRIEF DESCRIPTION OF THE DRAWINGS

FIGS. 1A and B depict illustrative embodiments of Coomassie blue-stained SDS gels showing uninduced (UN) and induced (IN) cell lysates, protein molecular size marker (M) and different lanes showing different elution fractions containing purified protein (lanes 1-7 in FIG. 1A and, lanes 1-5 in FIG. 1B) obtained during purification of Mycobacterium tuberculosis proteins, Rv1168c (FIG. 1A) and heat shock protein 60 (Hsp60) (FIG. 1B). The arrow on the right in FIG. 1 A indicates the position of the Rv1168c protein (˜42 kDa). The arrow on the right in FIG. 1B indicates the position of the mycobacterial Hsp60 protein (˜60 kDa), Both recombinant proteins were expressed in strain BL21 of Escherichia coli and were purified to homogeneity using the Ni-NTA protein purification kit.

FIGS. 2A-2C show illustrative embodiments of the discrimination of TB patients from BCC-vaccinated controls using the PPE protein Rv1168c. FIG. 2A shows an illustrative embodiment of a scatter plot of the sera cross-reactivity to the M. tuberculosis recombinant proteins Rv1168c, ESAT-6, Hsp60 and PPD in sera of either active tuberculosis patients or BCG-vaccinated controls as measured by enzyme immunoassay (EIA). The horizontal line indicates the mean of the absorbance values. In FIG. 2B, the Rv1168c and T-Hsp60 antibody response of individual patients, included in the results of FIG. 2A and as measured by EIA, is compared. In FIG. 2C, responders to Rv1168c were compared with that of E SAT-6, Hsp60 and PPD by calculating the percentage of TB patients showing absorbance value greater than or equal to the cutoff value calculated as mean OD₄₉₂ of control sera plus 6 SD. Mean OD₄₉₂ (SD) used for cutoff determinations were as follows: Rv1168c, 0.376 (0.066); ESAT-6, 0.343 (0.07), Hsp60, 0.359 (0.08) and PPD, 0.295 (0.071). Statistical significance was determined by student's t-test.

FIG. 3. shows illustrative embodiments of EIA-absorbance values at 492 nm from FIG. 2A replotted to compare Rv1168c-specific immune responses of pulmonary and extrapulmonary TB patients with that of BCG-vaccinated controls. Statistical significance was determined by ANOVA.

### DETAILED DESCRIPTION

In the following detailed description, reference is made to the accompanying drawings, which form a part hereof. In the drawings, similar symbols typically identify similar components, unless context dictates otherwise. The illustrative embodiments described in the detailed description, drawings, and claims are not meant to be limiting. Other embodiments may be utilized and other changes may be made, without departing from the spirit or scope of the subject matter presented here.

### DEFINITIONS

It is to be noted that the term “a” or “an” entity refers to one or more of that entity; for example, “a polypeptide,” is understood to represent one or more polypeptides. As such, the terms “a” (or “an”), “one or more,” and “at least one” can be used interchangeably herein.

As used herein, the term “polypeptide” is intended to encompass a singular “polypeptide” as well as plural “polypeptides,” and refers to a molecule composed of monomers (amino acids) linearly linked by amide bonds (also known as peptide bonds). The term “polypeptide” refers to any chain or chains of two or more amino acids, and does not refer to a specific length of the product. Thus, “peptides,” “dipeptides”, “tripeptides”, “oligopeptides”, “protein,” “amino acid chain,” or any other term used to refer to a chain or chains of two or more amino acids, are included within the definition of “polypeptide,” and the term “polypeptide” may be used instead of, or interchangeably with any of these terms. The term “polypeptide” is also intended to refer to the products of post-expression modifications of the polypeptide, including without limitation glycosylation, acetylation, phosphorylation, amidation, derivatization by known protecting/blocking groups, proteolytic cleavage, or modification by non-naturally occurring amino acids. A polypeptide may be derived from a natural biological source or produced by recombinant technology, but is not necessarily translated from a designated nucleic acid sequence. It may be generated in any manner, including by chemical synthesis.

An “antibody” or “antibody molecule,” as described herein, refers to a full-length (i.e., naturally occurring or formed by normal immunoglobulin gene fragment recombinatorial processes) immunoglobulin molecule (e.g., an IgG antibody) or an immunologically active (i.e., specifically binding) portion of an immunoglobulin molecule, like an antibody fragment.

The present technology includes certain Rv1168c polypeptide binding agents which bind an Rv1168c polypeptide including, but not limited to, antibodies, antibody or antigen-binding fragments, variants or derivatives thereof. Unless specifically referring to full-size antibodies, as described above, the term “polypeptide binding agent” encompasses full-sized antibodies as well as fragments, variants, analogs or derivatives of such antibodies, e.g., naturally occurring antibody or immunoglobulin molecules or engineered antibody molecules or fragments that bind antigen in a manner similar to antibody molecules—Fab fragments, scFv molecules, etc. Antibody fragments, including single-chain antibodies, may comprise the variable region(s) alone or in combination with the entirety or a portion of the following: hinge region, CH1, CH2, and CH3 domains. Also included in the present technology are antigen-binding fragments also comprising any combination of variable region(s) with a hinge region, CH1, CH2, and CH3 domains.

Polypeptide binding agents, as used herein, refers to a portion of an antibody such as F(ab′)₂, F(ab)₂, Fab′, Fab, Fv, scFv and the like. Regardless of structure, an antibody fragment binds with the same antigen that is recognized by the intact antibody. The term “polypeptide binding agent” includes, but is not limited to, aptamers, speigelmers, and diabodies. The term “polypeptide binding agent” also includes, but is not limited to, any synthetic or genetically engineered protein that acts like an antibody by binding to a specific antigen to form a complex. For example, antibody fragments include isolated fragments consisting of the variable regions, such as the “Fv” fragments consisting of the variable regions of the heavy and light chains, recombinant single chain polypeptide molecules in which light and heavy variable regions are connected by a peptide linker (“scFv proteins”), scFv HSA fusion polypeptides in which the scFv is expressed as a fusion to either the N or C terminus of HSA, Fab′ HSA fusion polypeptides in which the VH-CH1 or VK-CK are produced as a fusion to HSA, which then folds with its cognate VK-CK light chain or VH-CH1 heavy chain, respectively, to form a Fab′, and minimal recognition units consisting of the amino acid residues that mimic the hypervariable region.

A polypeptide binding agent, as used herein, comprises at least the variable domain of a heavy chain, and normally comprises at least the variable domains of a heavy chain and a light chain. Basic immunoglobulin structures in vertebrate systems are relatively well understood. See, e.g., Harlow et al., Antibodies: A Laboratory Manual, (Cold Spring Harbor Laboratory Press, 2nd ed. 1988).

The term “antigen-binding polypeptide” comprises various broad classes of polypeptides that can be distinguished biochemically. Those skilled in the art will appreciate that heavy chains are classified as gamma, mu, alpha, delta, or epsilon with some subclasses among them (e.g., γ 1-γ4) It is the nature of this chain that determines the “class” of the antibody as IgG, IgM, IgA IgG, or IgE, respectively. The immunoglobulin subclasses (isotypes), e.g., IgG1, IgG2, IgG3, IgG4, IgG5, etc. are well characterized and are known to confer functional specialization. Modified versions of each of these classes and isotypes are readily discernable to the skilled artisan in view of the instant disclosure and, accordingly, are within the scope of the present technology. Although all immunoglobulin classes are clearly within the scope of the present technology, the following discussion will generally be directed to the IgG class of immunoglobulin molecules. With regard to IgG, a standard immunoglobulin molecule comprises two identical light chain polypeptides of molecular weight approximately 23,000 Daltons, and two identical heavy chain polypeptides of molecular weight 53,000-70,000. The four chains are typically joined by disulfide bonds in a “Y” configuration wherein the light chains bracket the heavy chains starting at the mouth of the “Y” and continuing through the variable region.

Both the light and heavy chains are divided into regions of structural and functional homology. The terms “constant” and “variable” are used functionally. In this regard, it will be appreciated that the variable domains of both the light (VK) and heavy (VH) chain portions determine antigen recognition and specificity. Conversely, the constant domains of the light chain (CK) and the heavy chain (CH1, CH2 or CH3) confer important biological properties such as secretion, transplacental mobility, Fc receptor binding complement binding, and the like. By convention the numbering of the constant region domains increases as they become more distal from the antigen-binding site or amino-terminus of the antibody. The N-terminal portion is a variable region and at the C-terminal portion is a constant region; the CH3 and CK domains actually comprise the carboxy-terminus of the heavy and light chain, respectively.

As used herein, when an antibody or polypeptide binding agent “binds” to an Rv1168c polypeptide it is generally meant that the antibody or polypeptide binding agent binds to an epitope via its antigen-binding domain, and that the binding entails some complementarity between the antigen-binding domain and the epitope of the Rv1168c polypeptide. According to this definition, an antibody or polypeptide binding agent is said to “specifically bind” to an epitope when it binds to that epitope, via its antigen-binding domain more readily than it would bind to a random, unrelated epitope. The term “specificity” is used herein to qualify the relative affinity by which a certain antigen-binding polypeptide binds to a certain epitope. For example, antibody “A” may be deemed to have a higher specificity for a given epitope than antibody “B,” or antibody “A” may be said to bind to epitope “C” with a higher specificity than it has for related epitope “D.”

By way of non-limiting example, an antibody may be considered to bind a first epitope specifically if it binds a first epitope with a dissociation constant (K_{D}) that is less than the antibody's K_{D} for the second epitope. In another non-limiting example, an antibody may be considered to bind a first antigen specifically if it binds the first epitope with an affinity that is at least one order of magnitude less than the antibody's K_{D} for the second epitope. In another non-limiting example, an antibody may be considered to bind a first epitope specifically if it binds the first epitope with an affinity that is at least two orders of magnitude less than the antibody's K_{D} for the second epitope.

In another non-limiting example, an antibody may be considered to bind a first epitope specifically if it binds the first epitope with an off rate (k(off)) that is less than the antibody's k(off) for the second epitope. In another non-limiting example, an antibody may be considered to bind a first epitope specifically if it binds the first epitope with an affinity that is at least one order of magnitude less than the antibody's k(off) for the second epitope. In another non-limiting example, an antibody may be considered to bind a first epitope specifically if it binds the first epitope with an affinity that is at least two orders of magnitude less than the antibody's k(off) for the second epitope.

An antibody or or antigen-binding fragment, variant, or derivative disclosed herein may be said to bind a target polypeptide disclosed herein or a fragment or variant thereof with an off rate (k(off)) of less than or equal to 10⁻²sec⁻¹ to 10⁻⁷sec⁻¹, 10⁻²sec⁻¹ to 10⁻⁶sec⁻¹, 10⁻²sec⁻¹ to 10⁻⁵sec⁻¹, 10⁻²sec⁻¹ to 10⁻⁴sec⁻¹, 10⁻²sec⁻¹ to 10⁻³sec⁻¹, 5×10⁻²sec⁻¹, 10⁻²sec⁻¹, 5×10⁻³sec⁻¹, 10⁻³sec⁻¹, 5×10⁻⁴sec⁻¹, 10⁻⁴sec⁻¹, 5×10⁻⁵sec⁻¹, 10⁻⁵sec⁻¹, 5×10⁻⁶sec⁻¹, 10⁻⁶sec⁻¹, 5×10⁻⁷sec⁻¹ or 10⁻⁷sec⁻¹.

An antibody or or antigen-binding fragment, variant, or derivative disclosed herein may be said to bind a target polypeptide disclosed herein or a fragment or variant thereof with an on rate (k(on)) of greater than or equal to 10³M⁻¹sec⁻¹ to 10⁷M⁻¹sec⁻¹, 10³M⁻¹sec⁻¹ to 10⁶M⁻¹sec⁻¹. 10³M⁻¹sec⁻¹ to 10⁵M⁻¹sec⁻¹, 10³M⁻¹sec⁻¹ to 10⁴M⁻¹sec⁻¹, 10³M⁻¹sec⁻¹, 5×10³M⁻¹sec⁻¹, 10⁴M⁻¹sec⁻¹, 5×10⁴M⁻¹sec⁻¹, 10⁵M⁻¹sec⁻¹, 5×10⁵M⁻¹sec⁻¹, 10⁶M⁻¹sec⁻¹, 5×10⁶M⁻¹sec⁻¹ or 10⁷M⁻¹sec⁻¹.

Antibodies or polypeptide binding agents for use in the methods of the present technology may also be described or specified in terms of their binding affinity to a Rv1168c polypeptide, or fragment thereof. Suggested binding affinities include those with a dissociation constant or K_{D} less than 10⁻²M to 10⁻¹⁵M, 10⁻²M to 10⁻¹⁴M, 10⁻²M to 10⁻¹³M, 10⁻²M to 10⁻¹²M, 10⁻²M to 10⁻¹¹M, 10⁻²M to 10⁻¹⁰M, 10⁻²M to 10⁻⁹M, 10⁻²M to 10⁻⁸M, 10⁻²M to 10⁻⁷M, 10⁻²M to 10⁻⁶M, 10⁻²M to 10⁻⁵M, 10⁻²M to 10⁻⁴M, 10⁻²M to 10⁻³M, 5×10⁻² M, 10⁻²M, 5×10⁻³M, 10⁻³M, 5×10⁻⁴M, 10⁻⁴M, 5×10⁻⁵M, 10⁻⁵M, 5×10⁻⁶M, 10⁻⁶M, 5×10⁻⁷M, 10⁻⁷M, 5×10⁻⁸M, 10⁻⁸M, 5×10⁻⁹M, 10⁻⁹M, 5×10⁻¹⁰M, 10⁻¹⁰M, 5×10⁻¹¹M, 10⁻¹¹M, 5×10⁻¹²M, 10⁻¹²M, 5×10⁻¹³M, 10⁻¹³M, 5×10⁻¹⁴M, 10⁻¹⁴M, 5×10⁻¹⁵M, or 10⁻¹⁵M.

As used herein, the terms “linked,” “conjugated,” “futsed” or “fusion” are used interchangeably. These terms refer to the joining together of two more elements or components, by whatever means including chemical conjugation or recombinant means. A fusion protein refers to a single protein containing two or more segments that correspond to polypeptides which are not normally so Joined in nature. The segments may be physically or spatially separated by, for example, a linker polypeptide sequence.

The methods of the present technology also encompass variants of the Rv1168c polypeptides and polypeptide binding agents. A “variant,” as used herein, is a polypeptide or polypeptide binding agent that differs from the reference polypeptide or polypeptide binding agent in amino acid substitutions and/or modifications, such that the ability to form Rv1168c polypeptide-antibody or Rv1168c polypeptide binding agent-Rv1168c polypeptide complexes is maintained. Variants for use in the methods of the present technology may include, but are not limited to, polypeptides or polypeptide binding agents containing conservative substitutions. As used herein, a “conservative substitution” is one in which an amino acid is substituted for another amino acid that has similar properties, such that one skilled in the art of peptide chemistry would expect the secondary structure and hydropathic nature of the polypeptide to be substantially unchanged. In general, any of the amino acids in the following groups may be substituted for another amino acid member of the same group as a conservative substitution: (1) ala pro, gly, glu, asp, gin, asn, ser, thr, (2) cys, ser, tyr, thr; (3) val, ile, leu, met, ala, phe; (4) lys, arg, his; and (5) phe, tyr, trp, his.

Variants include polypeptides and polypeptide binding agents at least about 70%, at least about 75%, at least about 80%, at least about 85%, at least about 90%, at least about 95%, at least about 96%, at least about 97%, at least about 98% and at least about 99% to the reference polypeptides. One of skill in the art would be able to determine if a variant Rv1168c polypeptide or polypeptide binding agent is useful in the methods of the present technology utilizing any of the assays described herein.

By “subject” or “individual” or “animal” or “patient” or “mammal” is meant any subject, for example, a mammalian subject, for whom diagnosis, detection, prognosis, or therapy is desired. Mammalian subjects include but are not limited to humans, domestic animals, farm animals, and zoo, sport, or pet animals such as dogs, cattle, swine and other such animals which are infected by Mycobacterium tuberculosis.

### The Rv1168C Polypeptide

Rv1168c polypeptides, or fragments thereof for use in the methods of the present technology include polypeptides, or fragments thereof, which comprise, consist essentially of, or consist of the amino acid sequence of SEQ ID NO:1 The Rv1168c polypeptides, or fragments thereof, may be used in the assays described herein to detect the presence of M. tuberculosis Rv1168c antibodies in a test biological sample.

The Rv1168c polypeptide is a member of the PPE gene family of M. tuberculosis. Gey van Pittius N. C., et al., BMC Evol. Biol. 6:95 (2006) (19). The PPE gene family encodes 69 different polypeptides with mostly unknown function. Id. The polypeptides in the PPE family contain a highly conserved and unique N-terminal domain of approximately 180 amino acids with a Proline-Proline-Glutamic Acid (PPE) motif at amino acid positions 7-9. Id. The PPE family has been divided into four subfamilies of which the PPE-SVP subfamily is the largest (24 polypeptides), Id. The polypeptides in this subfamily are characterized by the motif Gly-X-X-Ser-Val-Pro-X-X-Trp (SEQ ID NO: 17). Id. The Rv1168c polypeptide is a member of the PPE-SVP subfamily. Id.

The Rv1168c polypeptide is also known by the name PPE17 or MT1205, The polypeptide is 346 amino acids in length and the amino acid sequence for the Rv1168c polypeptide can be found at UniProtKB/TrEMBL accession number Q7D8Q2 and GenBank Accession No YP₋177791, which both are hereby incorporated by reference in their entireties. The amino acid sequence for the Rv1168c polypeptide is reproduced below:

(SEQ ID NO: 1)

MDFTIFPPEF NSLNIQGSAR PFLVAANAWK NLSNELSYAA
SRFESEINGL ITSWRGPSST IMAAAVAPFR AWIVTTASLA
ELVADHISVV AGAYEAAHAA HVPLPVEITN RLTRLALATT
NIFGIHTPAI FALDALYAQY WSQDGEAMNL YATMAAAAAR
LTPFSPPAPI ANPGALARLY ELIGSVSETV GSFAAPATKN
LPSKLWTLLT KGTYPLTAAR ISSIPVEYVL AFVEGSNMGQ
MMGNLAMRSL TPTLKGPLEL LPNAVRPAVS ATLGNADTIG
GLSVPPSWVA DKSITPLAKA VPTSAPGGPS GTSWAQLGLA
SLAGGAVGAV AARTRSGVIL RSPAAG.

The nucleic acid sequence which encodes the Rv1168c polypeptide is 1041 nucleotides in length and can be found at the GenBank Accession No. NC_{—}002755, which is incorporated herein by reference in its entirety. The nucleic acid sequence encoding Rv1168c is reproduced below:

(SEQ ID NO: 2)

atggatttca caatttttcc gccggagttc aactccctca
acatccaagg tagcgctcgt ccgtttctag tagccgcgaa
cgcctggaag aatctgtcca acgagctgag ctacgcggcc
agtcggttcg agagtgagat caacgggctg atcacatcgt
ggcgggggcc atcgtcgacg atcatggcag ctgcggtcgc
cccatttcgg gcctggattg tcacgaccgc ttccctggct
gaactcgtcg ccgaccacat cagcgtcgtg gcaggcgcct
atgaagcggc gcacgcagca cacgtgccgc tgccggtgat
cgagaccaac cgactgacgc gcctcgctct cgccacgacc
aacattttcg ggattcacac ccccgcgatc tttgccctcg
atgcactgta tgcccagtac tggtcccaag atggcgaggc
gatgaacctc tacgccacaa tggcggcggc cgccgcacgg
ctgacaccgt tctcgccccc ggcgccgatc gccaacccgg
gcgcgctggc cagactttat gaactgatcg gttcggtgtc
cgagacggtg gggtcattcg ccgcgccggc gaccaagaat
ctgccttcga agctgtggac gctgttgacg aagggcacct
acccgctcac agccgcgcga atctcgtcga tacccgtgga
atacgtgttg gcctttgtcg agggcagcaa catgggccag
atgatgggca acctcgccat gcggagcctg acacccacgc
tcaagggccc gctggagttg ctacccaacg cggtcaggcc
cgcggtgtcg gcaacattgg gaaatgcgga tacgatcggg
gggttgtcgg tgccccccag ctgggttgcg gacaaatcga
ttacgccgtt ggccaaagcc gtcccgacct ccgcgccggg
cggtccgtcg ggaacctcgt gggcccagct gggattggca
agcctggccg ggggcgctgt gggcgccgtc gcggcaagaa
cccgttccgg agtgatactg cggtcacccg ccgccggcta g.

Additional Rv1168c polypeptides for use in the methods of the present technology include fragments of Rv1168c, e.g., antigenic Rv1168c fragments, which comprise, consist essentially of, or consist of at least about 4 to 100, 4 to 75, 4 to 50, 4 to 45, 4 to 40, 4 to 35, 4 to 30, 4 to 25, 4 to 2, 4 to 19, 4 to 18, 4 to 17, 4 to 16, 4 to 15, 4 to 14, 4 to 13, 4 to 12, 4 to 11, 4 to 10, at least about 4, about 5, about 6, about 7, about 8, about 9, about 10, about 11, about 12, about 13 about 14, about 15, about 16, about 17, about 18, about 19, about 20, about 21, about 22, about 23, about 24, about 25 about 30, about 35, about 40, about 45, about 50, about 55, about 60, about 65, about 70, about 75, about 80, about 85, about 90, about 95, about 100 contiguous or non-continuous amino acids of SEQ ID NO: 1, where the non-contiguous amino acids form an epitope through protein folding, Rv1168c polypeptide fragments may be any length including, but not limited to, the lengths described above. Fragments of the Rv1168c polypeptide may be used in any of the methods described herein in combination with the Rv1168c polypeptide of SEQ ID NO: 1 or other antigenic M. tuberculosis polypeptides.

Non-limiting examples of Rv1168c polypeptide fragments include those fragments comprising, consisting essentially of, or consisting of amino acids 10 to 18 of SEQ ID NO: 1; amino acids 29 to 35 of SEQ ID NO: 1; amino acids 37 to 46 of SEQ ID NO: 1; amino acids 52 to 59 of SEQ ID NO: 1 amino acids 109 to 114 of SEQ ID NO: 1; amino acids 138 to 149 of SEQ ID NO: 1 amino acids 196 to 203 of SEQ ID NO:1; amino acids 213 to 216 of SEQ ID NO:1; amino acids 236 to 241 of SEQ ID NO: 1; amino acids 251 to 256 of SEQ ID NO: 1; amino acids 288 to 292 of SEQ ID NO: 1 and amino acids 305 to 314 of SEQ ID NO:1 The fragments of Rv1168c described above may include one or more additional amino acids on either end of the fragments described.

The Rv1168c polypeptide, and fragments thereof for use in the methods of the present technology can be prepared by any method known to one of skill in the art. Non-limiting examples of techniques to prepare the Rv1168c polypeptide are discussed below.

Rv1168c polypeptide, and fragments thereof, may be produced recombinantly using a DNA sequence that encodes the polypeptide (e.g., SEQ ID NO:2), which has been inserted into an expression vector and expressed in an appropriate host using techniques well known in the art, such as those described in Sambrook et al., Molecular Cloning: A Laboratory Manual, Cold Spring Harbor Laboratories, Cold Spring Harbor, N.Y., 1989.

DNA sequences encoding Rv1168c, and fragments thereof, may be obtained from M. tuberculosis cDNA or genomic DNA or from patients infected with M. tuberculosis using the polymerase chain reaction (PCR) and oligonucleotides specific for Rv1168c using methods well known in the art.

Recombinant Rv1168c polypeptides, fragments and/or variants thereof may be readily prepared from a DNA sequence encoding the polypeptide using a variety of techniques well known to those of ordinary skill in the art. For example, supernatants from suitable host/vector systems which secrete recombinant protein into culture media may be first concentrated using a commercially available filter. Following concentration, the concentrate may be applied to a suitable purification matrix such as, but not limited to, an affinity matrix or an ion exchange resin, Finally one or more reverse phase HPLC steps can be employed to further purity a recombinant protein.

Any of a variety of expression vectors known to those of ordinary skill in the art may be employed to express recombinant polypeptides as described herein. The following vectors are provided by way of example. Useful bacterial vectors include, but are not limited to, phagescript, PsiX174, pBluescript SK, pBS KS, pNH8a, pNH16a, pNH18a, pNH46a (available from Stratagene); pTrc99A, pKK223-3, pKK233-3, pDR540, pRIT5 (Pharmacia, Uppsala, Sweden), and pRSET-A (available from Invitrogen). Useful eukaryotic vectors include, but are not limited to, pWLneo, pSV2cat, pOG44, pXT1, pSG (available from Stratagene) pSVK3, pBPV, pMSG, pSVL (Pharmacia, Uppsala, Sweden) and pQE (Qiagen, Valencia, Calif., USA).

Expression may be achieved in any appropriate host cell that has been transformed or transfected with an expression vector containing a DNA molecule that encodes a recombinant polypeptide Suitable host cells include, but are not limited to, prokaryotes, yeast, insects and higher eukaryotic cells. In illustrative embodiments, the host cells employed are E. coli, yeast or a mammalian cell line, such as COS or CHO. The DNA sequences expressed in this manner may encode Rv1168c polypeptides, fragments, or other variants thereof.

Antigenic fragments of M. tuberculosis Rv1168c polypeptide may also be prepared and identified using well known techniques, such as those summarized in Paul, Fundamental Immunology, 3d ed., Raven Press, 1993, pp. 243 247 Such techniques include, but are not limited to, screening polypeptide portions of the Rv1168c for antigenic properties. ELISAs, as described herein, or other similar assays may generally be employed in these screens. An antigenic fragment of an Rv1168c polypeptide is a portion that, within such representative assays, generates a signal in such assays that is substantially similar to that generated by the full length Rv1168c antigen. In other words, an antigenic fragment of a Rv1168c polypeptide generates at least about 20%-100%, about 75%-100%, about 80%, about 85%, about 90%, about 95% and about 100% of the signal induced by the full length antigen in a model ELISA, or other similar assay, as described herein.

Rv1168c polypeptides, fragments and other variants may be generated by synthetic or recombinant means. Synthetic polypeptides having fewer than about 100 amino acids, and fewer than about 50 amino acids, may be generated using techniques well known in the art. For example, such polypeptides may be synthesized using any of the commercially available solid-phase techniques, such as, but not limited to, the Merrifield solid-phase synthesis method, where amino acids are sequentially added to a growing amino acid chain. See Merrifield, J. Am. Chem. Soc. 85:2149 2146, 1963. Equipment for automated synthesis of polypeptides is commercially available from suppliers such as Applied BioSystems. Inc., Foster City, Calif., and may be operated according to the manufacturer's instructions.

Variants of an Rv1168c polypeptide may generally be prepared using standard mutagenesis techniques, such as, but not limited to, oligonucleotide-directed site-specific mutagenesis. Sections of the DNA sequence may also be removed using standard techniques to permit preparation of truncated polypeptides.

In general, regardless of the method of preparation, the polypeptides disclosed herein are prepared in substantially pure form. “Substantially pure,” as used herein, refers to a purified or isolated polypeptide, or a substantially pure preparation of a polypeptide, that has been separated from other proteins, lipids, and nucleic acids with which it naturally occurs and/or substances which are used to purify it, e.g., antibodies or gel matrix such as polyacrylamide. Protein purification techniques are well known in the art and may rely upon the addition of a purification facilitating polypeptide, as described herein, fused to Rv1168c polypeptides.

### Rv1168c Antigen Binding Agents

Rv1168c polypeptide binding agents, or fragments thereof, for use in the methods of the present technology include, but are not limited to, antibodies, or fragments thereof, which bind to the Rv1168c polypeptide, The Rv1168c polypeptide binding agents, or fragments thereof, may be used in the assays described herein to detect the presence of M. tuberculosis Rv1168c polypeptide in a test biological sample.

Antigen binding agents, and specifically antibodies, for use in the methods of the present technology may be prepared by any of a variety of techniques known to those of ordinary skill in the art. Monoclonal antibodies can be prepared using a wide variety of techniques known in the art including the use of hybridoma, recombinant, and phage display technologies, or a combination thereof. For example, monoclonal antibodies can be produced using hybridoma techniques including those known in the art and taught, for example, in Harlow et al., Antibodies: A Laboratory Manual, Cold Spring Harbor Laboratory Press, 2nd ed. (1988); Hammerling et al., in: Monoclonal Antibodies and T-Cell Hybridomas Elsevier, N.Y., 563-681 (1981). The term “monoclonal antibody” as used herein is not limited to antibodies produced through hybridoma technology. The term “monoclonal antibody” refers to an antibody that is derived from a single clone, including any eukaryotic, prokaryotic, or phage clone, and not the method by which it is produced.

Alternatively, techniques described for the production of single chain antibodies (U.S. Pat. No. 4,694,778; Bird, Science 242:423-442 (1988); Huston et al., Proc. Natl. Acad. Sci. USA 55:5879-5883 (1988); and Ward et al., Nature 334:544-554 (1989) can be adapted to produce single chain antibodies for use in the methods of the present technology. Single chain antibodies are formed by linking the heavy and light chain fragments of the Fv region via an amino acid bridge, resulting in a single chain antibody. Techniques for the assembly of functional Fv fragments in E. coli may also be used (Skerra et al., Science 242: 1038-1041 (1988).

Additionally, antibody fragments for example, Fab and F(ab′)2 fragments, may be generated by known techniques including proteolytic cleavage of immunoglobulin molecules, using enzymes such as papain (to produce Fab fragments) or pepsin (to produce F(ab′)2 fragments), F(ab′)2 fragments contain the variable region, the light chain constant region and the CH1 domain of the heavy chain.

Examples of techniques which can be used to produce single-chain Fvs (scfvs) and antibodies include those described in U.S. Pat. Nos. 4,946,778 and 5,258,498; Huston et al., Methods in Enzymology 203:46-88 (1991); Shu et al., Proc. Natl. Sci. USA 90:1995-1999 (1993); and Skerra et al., Science 240:1038-1040 (1988). For some uses, chimeric, humanized, or human antibodies may be used. A chimeric antibody is a molecule in which different portions of the antibody are derived from different animal species, such as antibodies having a variable region derived from a murine monoclonal antibody and a human immunoglobulin constant region. Methods for producing chimeric antibodies are known in the art. See, e.g., Morrison, Science 229:1202 (1985); Oi et al., BioTechniques 4:214 (1986); Gillies et al., J. Immunol. Methods 125:191-202 (1989); U.S. Pat. Nos. 5,807,715; 4,816,567; and 4,816,397.

Humanized antibodies are antibody molecules derived from a nonhuman species antibody that bind the desired antigen having one or more complementarity determining regions (CDRs) from the non-human species aid framework regions from a human immunoglobulin molecule. Often, framework residues in the human framework regions will be substituted with the corresponding residue from the CDR donor antibody to alter, and improve, antigen-binding. These framework substitutions are identified by methods well known in the art, e.g., by modeling of the interactions of the CDR and framework residues to identify framework residues important for antigen-binding and sequence comparison to identify unusual framework residues at particular positions. (See, e.g., Queen et al., U.S. Pat. No. 5,585,089; Riechmann et al., Nature 332:323 (1988).) Antibodies can be humanized using a variety of techniques known in the art including, for example, CDR-grafting (EP 239,400; PCT publication WO 91/09967; U.S. Pat. Nos. 5,225,539; 5,530,101; and 5,585,089), veneering or resurfacing (EP 592,106; EP 519,596; Padlan, Molecular Immunology 28 (4/5): 489-498 (1991); Studnicka et al., Protein Engineering 7(6):805-814 (1994); Roguska, et al., Proc. Natl. Sci. USA 91:969-973 (1994)), and chain shuffling (U.S. Pat. No. 5,565,332). Human antibodies can be made by a variety of methods known in the art including phage display methods using antibody libraries derived from human immunoglobulin sequences. See also, U.S. Pat. Nos. 4,444,887 and 4,716,111; and PCT publications WO 98146645, WO 98/50433, WO 98/24893, WO 98/16654, WO 96/34096, WO 96/33735, and WO 91/10741.

Human antibodies can also be produced using transgenic mice which are incapable of expressing functional endogenous immunoglobulins, but which can express human immunoglobulin genes. For an overview of this technology for producing human antibodies, see Lonberg and Huszar Int. Rev. Immunol. 73:65-93 (1995). For a detailed discussion of this technology for producing human antibodies and human monoclonal antibodies and protocols for producing such antibodies, see, e.g., PCT publications WO 98/24893; WO 96/34096; WO 96/33735; U.S. Pat. Nos. 5,413,923; 5,625,126; 5,633,425; 5,569,825; 5,661,016; 5,545,806; 5,814,318; and 5,939,598. In addition, companies such as Abgenix, Inc. (Freemont, Calif., USA) and GenPharm (San Jose, Calif., USA) can be engaged to provide human antibodies directed against a selected antigen using technology similar to that described above.

Rv1168c polypeptide binding agents for use in the methods of the present technology may also be produced recombinantly. DNA encoding desired monoclonal antibodies may be readily isolated and sequenced using conventional procedures (e.g., by using oligonucleotide probes that are capable of binding specifically to genes encoding the heavy and light chains of murine antibodies). The isolated and subcloned hybridoma cells serve as a source of such DNA. Once isolated, the DNA may be placed into expression vectors, which are then transfected into prokaryotic or eukaryotic host cells such as E. coli cells, simian COS cells, Chinese Hamster Ovary (CHO) cells or myeloma cells that do not otherwise produce antibodies. More particularly, the isolated DNA (which may be synthetic as described herein) may be used to clone constant and variable region sequences for the manufacture antibodies as described in Newman et al., U.S. Pat. No. 5,658,570, filed Jan. 25, 1995. Essentially, this entails extraction of RNA from the selected cells, conversion to cDNA, and amplification by PCR using Ig specific primers. Suitable primers for this purpose are also described in U.S. Pat. No. 5,658,570. As will be discussed in more detail below, transformed cells expressing the desired antibody may be grown up in relatively large quantities to provide clinical and commercial supplies of the immunoglobulin.

Additionally, using routine recombinant DNA techniques, one or more of the CDRs of the antigen-binding polypeptides of the present technology, may be inserted within framework regions. e.g., into human framework regions to humanize a non-human antibody. The framework regions may be naturally occurring or consensus framework regions. In some embodiments, the framework regions are human framework regions (see, e.g., Chothia et al., J. Mol. Biol. 278:457-479 (1998) for a listing of human framework regions). In illustrative embodiments, the polynucleotide generated by the combination of the framework regions and CDRs encodes an antibody that specifically binds to at least one epitope of a desired polypeptide, e.g., Rv1168c. In some embodiments, one or more amino acid substitutions may be made within the framework regions. In illustrative embodiments, the amino acid substitutions improve binding of the antibody to its antigen. Additionally, such methods may be used to make amino acid substitutions or deletions of one or more variable region cysteine residues participating in an intrachain disulfide bond to generate antibody molecules lacking one or more intrachain disulfide bonds. Other alterations to the polynucleotide are encompassed by the present technology and within the skill of the art.

In addition, techniques developed for the production of “chimeric antibodies” (Morrison et al., Proc. Natl. Acad. Sci. USA: 851-855 (1984); Neuberger et al., Nature 372:604-608 (1984); Takeda et al., Nature 314:452-454 (1985)) by splicing genes from a mouse antibody molecule, of appropriate antigen specificity, together with genes from a human antibody molecule of appropriate biological activity can be used. As used herein, a chimeric antibody is a molecule in which different portions are derived from different animal species, such as those having a variable region derived from a murine monoclonal antibody and a human immunoglobulin constant region.

Antibody-producing cell lines may be selected and cultured using techniques well known to the skilled artisan. Such techniques are described in a variety of laboratory manuals and primary publications. In this respect, techniques suitable for use in the present technology as described below are described in Current Protocols in Immunology, Coligan et al., Eds., Green Publishing Associates and Wiley-Interscience, John Wiley and Sons, New York (1991).

Antibodies may be used in the methods of the present technology to detect exposure to or infection by M. tuberculosis using assays described herein and other techniques well known to those of skill in the art.

### Fusion and Conjugated Polypeptides or Antigen Binding Agents

Rv1168c polypeptides and polypeptide binding agents for use in the methods of the present technology may further be recombinantly fused to a heterologous polypeptide at the N- or C-terminus or chemically conjugated (including covalent and non-covalent conjugations) to polypeptides or other compositions. For example, Rv1168c polypeptides may be recombinantly fused or conjugated to molecules useful as labels in detection assays and effector molecules such as heterologous polypeptides, drugs, radionuclides, or toxins. See, e.g., PCT publications WO 92/08495; WO 91/14438; WO 89/12624; U.S. Pat. No. 5,314,995; and EP 396,387. Additionally, Rv1168c polypeptides may be recombinantly fused or conjugated to molecules useful for purification such as a purification facilitating polypeptide.

Rv1168c polypeptides of antigen-binding agents for use in the methods of the present technology include derivatives that are modified, i.e., by the covalent attachment of any type of molecule to the polypeptide or antigen-binding agent such that covalent attachment does not prevent the polypeptide from binding Rv1168c antibodies or the Rv1168c polypeptide binding agent from binding to Rv1168c antigen. For example, but not by way of limitation, the Rv1168c polypeptide and polypeptide binding agent derivatives include polypeptides and antibodies that have been modified, e.g., by glycosylation, acetylation, pegylation, phosphylation, phosphorylation, amidation, derivatization by known protecting/blocking groups, proteolytic cleavage, linkage to a cellular ligand or other protein, etc. Any of numerous chemical modifications may be carried out by known techniques, including, but not limited to specific chemical cleavage, acetylation, formylation, metabolic synthesis of tunicamycin, etc. Additionally, the derivative may contain one or more non-classical amino acids.

Rv1168c polypeptides or antigen-binding agents for use in the methods of the present technology can be composed of amino acids joined to each other by peptide bonds or modified peptide bonds, i.e., peptide isosteres, and may contain amino acids other than the 20 gene-encoded amino acids. Rv1168c polypeptides or antigen-binding agents may be modified by natural processes, such as posttranslational processing, or by chemical modification techniques which are well known in the art. Such modifications are well described in basic texts and in more detailed monographs, as well as in a voluminous research literature. Modifications can occur anywhere in the Rv1168c polypeptides or antigen-binding agents, including the peptide backbone, the amino acid side-chains and the amino or carboxyl termini, or on moieties such as carbohydrates. It will be appreciated that the same type of modification may be present in the same or varying degrees at several sites in a given Rv1168c polypeptide or polypeptide binding agents. Also, Rv1168c polypeptides or polypeptide binding agents may contain many types of modifications.

Additionally, Rv1168c polypeptides or polypeptide binding agents may be branched for example, as a result of ubiquitination, and they may be cyclic, with or without branching. Cyclic, branched, and branched cyclic Rv1168c polypeptides or polypeptide binding agents may result from posttranslation natural processes or may be made by synthetic methods. Modifications include, but are not limited to, acetylation, acylation, ADP-ribosylation, amidation, covalent attachment of flavin, covalent attachment of a heme moiety, covalent attachment of a nucleotide or nucleotide derivative, covalent attachment of a lipid or lipid derivative, covalent attachment of phosphotidylinositol, cross-linking, cyclization, disulfide bond formation, demethylation, formation of covalent cross-links, formation of cysteine, formation of pyroglutamate, formylation, gamma-carboxylation, glycosylation, GPI anchor formation, hydroxylation, iodination, methylation, myristoylation, oxidation, pegylation, proteolytic processing, phosphorylation, prenylation, racemization, selenoylation, sulfation, transfer-RNA mediated addition of amino acids to proteins such as arginylation, and ubiquitination. (See, for instance, Proteins-Structure And Molecular Properties, T. E. Creighton, W.H. Freeman and Company, New York 2nd Ed., (1993); Posttranslational Covalent Modification Of Proteins, B. C. Johnson, Ed., Academic Press, New York, pgs. 1-12 (1983); Seifter et al., Meth. Enzymol. 182:626-646 (1990); Rattan et al., Ann. NY. Acad. Sci. 663 S-62 (1992)).

The present technology also provides for fusion proteins for use in the present technology comprising an Rv1168c polypeptide or polypeptide binding agent and a heterologous polypeptide. The heterologous polypeptide to which the Rv1168c polypeptide or polypeptide binding agent is fused may be useful for synthesis, purification, identification or function, among others in methods disclosed herein. In illustrative embodiments, fusions may include fusions of the immunoglobulin Fc region, T cell receptor (Gascoigne et al., Proc. Natl. Acad. Sci. USA 54:2936-2940 (1987)); CD4 (Capon et al., Nature 337:525-531 (1989); Traunecker et al., Nature 339:68-70 (1989); Zettmeissl et al., DNA Cell Biol. USA: 347-353 (1990) and Byrn et al., Nature 344:667-670 (1990)); L-selectin (homing receptor) (Watson et al., J. Cell. Biol. 110:2221-2229 (1990); and Watson et al., Nature 349:164-167 (1991)); CD44 (Aruffo et al., Cell (57:1303-1313 (1990)), CD28 and B7 (Linsley e al., J. Exp. Med. 773:721-730 (1991)); CTLA-4 (Lisley et al., J. Exp. Med. 174:561-569 (1991)); CD22 (Stamenkovic et al., Cell 66: 1133-1144 (1991)); TNF receptor (Ashkenazi et al., Proc. Natl. Acad. Sci. USA 55:10535-10539 (1991); Lesslauer et al., Eur. J. Immunol. 27:2883-2886 (1991); and Peppel et al., Exp. Med. 774:1483-1489 (1991)); and IgE receptor a (Ridgway and Gorman, J. Cell. Biol. Vol. 115, Abstract No. 1448 (1991)).

Moreover, Rv1168c polypeptide or polypeptide binding agents can be fused to marker sequences, such as but not limited to a heterologous polypeptide, to facilitate purification or detection for example. Such purification or detection facilitation polypeptides, include but are not limited to, a poly-histidine tag, such as the tag provided in a pQE vector (QIAGEN, Inc., 9259 Eton Avenue, Chatsworth, Calif., 91311), among other vectors which are commercially available. As described in Gentz et al., Proc. Natl. Acad. Sci. USA 55:821-824 (1989), for instance, hexa-histidine (a peptide with a amino acids sequence of six histadines) provides for convenient purification of the fusion protein. Other peptide tags useful for purification include, but are not limited to, the “HA” tag, which corresponds to an epitope derived from the influenza hemagglutinin protein (Wilson et al., Cell 37:161 (1984)) and the “flag” tag.

Fusion proteins of the Rv1168c polypeptides and polypeptide binding agents described herein can be prepared using methods that are well known in the art (see for example U.S. Pat. Nos. 5,116,964 and 5,225,538). The precise site at which the fusion is made may be selected empirically to prevent disruption of the formation of Rv1168c polypeptide-Rv1168c antibody complexes or Rv1168c polypeptide binding agent-Rv1168c polypeptide complexes, DNA encoding the fusion protein is then transfected into a host cell for expression.

The present technology further encompasses Rv1168c polypeptides or antigen-binding agents conjugated to a detection agent for use in the assays described below, for example. Detection can be facilitated by coupling the Rv1168c polypeptides or antigen-binding agents to a detectable substance. Examples of detectable substances include, but are not limited to, various enzymes, prosthetic groups, fluorescent materials, luminescent materials, bioluminescent materials, radioactive materials, positron emitting metals using various positron emission tomographies, and nonradioactive paramagnetic metal ions, Non-limiting examples of suitable enzymes include horseradish peroxidase, alkaline phosphatase, beta-galactosidase, or acetylcholinesterase; examples of suitable prosthetic group complexes include, but are not limited to, streptavidin/biotin and avidin/biotin; examples of suitable fluorescent materials include, but are not limited to, umbelliferone, fluoresce in, fluorescein isothiocyanate, rhodamine, dichlorotriazinylamine fluorescein, dansyl chloride or phycoerythrin; an example of a luminescent material includes, but is not limited to, luminol; examples of bioluminescent materials include, but are not limited to, luciferase, luciferin, and aequorin; and examples of suitable radioactive materials include, but are not limited to, ¹²⁵I, ¹³¹I, ¹¹¹In or ⁹⁹Tc.

Those skilled in the art will appreciate that conjugates may also be assembled using a variety of techniques depending on the selected agent to be conjugated. For example, conjugates with biotin are prepared e.g., by reacting a binding polypeptide with an activated ester of biotin such as the biotin N-hydroxysuccinimide ester. Similarly, conjugates with a fluorescent marker may be prepared in the presence of a coupling agent, e.g., those listed herein, or by reaction with an isothiocyanate, such as, for example, fluorescein-isothiocyanate.

### Detection Assays

In another aspect, the present technology provides methods for using the polypeptides and polypeptide binding agents described above to detect exposure to M. tuberculosis or diagnose M. tuberculosis infection. In this aspect, methods are provided for detecting M. tuberculosis exposure or infection in a test biological sample, using a Rv1168c polypeptide or polypeptide binding agents or fragments thereof. Other known M. tuberculosis polypeptides may be used in the methods described herein such as the 38 kD antigen described in Andersen and Hansen, Infect. Immun. 57:2481 2488, 1989, culture filtrate protein 10 (CFP-10) described in Dillon et al., J. of Clinical Microbiol. 38:3285-3290 (2000), Purified Protein Derivative (PPD), Hsp60, ESAT-6 and M. tuberculosis polypeptides described in U.S. Pat. No. 7,122,196, and references 45, 46, 47 and 48.

As used herein a “biological sample” is any antibody-containing or protein-containing sample obtained from a patient. The sample may be whole blood, sputum, serum, plasma, saliva, cerebrospinal fluid or urine, The Rv1168c polypeptides or polypeptide binding agents are used in assays, described below, to determine the presence or absence of Rv1168c antibodies or proteins in the test biological sample, relative to a control sample. The presence or absence of Rv1168c antibodies or proteins in the test biological sample is usually determined based on a predetermined cut-off value which is calculated as described below. The presence of Rv1168c polypeptides or antibodies in the test biological sample is indicative of exposure to M. tuberculosis which may also be indicative of M. tuberculosis infection.

There are a variety of assay formats known to those of ordinary skill in the art for using polypeptides to detect antibodies in a sample or using an antigen binding agent to detect polypeptides in a sample. See, e.g., Harlow and Lane, Antibodies: A Laboratory Manual, Cold Spring Harbor Laboratory, 1988. In one example, the assay involves the use of an Rv1168c polypeptide or polypeptide binding agent immobilized on a solid support to bind to and remove the antibody or polypeptide from the sample. The bound Rv1168c polypeptide or antibody (i.e., the formation of a Rv1168c polypeptide-antibody or Rv1168c polypeptide binding agent-Rv1168c polypeptide complex) may then be detected using a detection reagent that contains a reporter group. Suitable detection reagents include, but are not limited to antibodies that bind to the antibody/polypeptide complex and free polypeptide labeled with a reporter group (e.g., in a semi-competitive assay). Alternatively, a competitive assay may be utilized, in which an antibody that binds to the polypeptide is labeled with a reporter group and allowed to bind to the immobilized antigen after incubation of the antigen with the sample. The extent to which components of the sample inhibit the binding of the labeled antibody to the polypeptide is indicative of the reactivity of the sample with the immobilized polypeptide.

The solid support may be any solid material known to those of ordinary skill in the art to which the Rv1168c polypeptide or polypeptide binding agent may be attached, For example, the so id support may be a test well in a microtiter plate or a nitrocellulose membrane or other suitable membranes. Alternatively, the support may be a bead or disc, such as, but not limited to, glass, fiberglass, latex or a plastic material such as polystyrene or polyvinylchloride. The support may also be a magnetic particle or a fiber optic sensor, such as those disclosed, for example, in U.S. Pat. No. 5,359,681.

The Rv1168c polypeptide or polypeptide binding agent may be bound to the solid support using a variety of techniques known to those of ordinary skill in the art, which are amply described in the patent and scientific literature. In the context of the present technology, the term “bound” refers to both noncovalent association, such as adsorption, and covalent attachment (which may be a direct linkage between the polypeptide or the antigen-binding agent and functional groups on the support or may be a linkage by way of a cross-linking agent). The Rv1168c polypeptide or polypeptide binding agent may also be bound by adsorption to a well in a microtiter plate or to a membrane. In such cases, adsorption may be achieved by contacting the Rv1168c polypeptide or polypeptide binding agent, in a suitable buffer, with the solid support for a suitable amount of time. The contact time varies with temperature, but is typically between about 1 hour and 1 day. In general, contacting a well of a plastic microtiter plate (such as polystyrene or polyvinylchloride) with an amount of polypeptide or polypeptide binding agent ranging from about 10 ng to about 1 mg, and about 100 mg, is sufficient to bind an adequate amount of polypeptide or antibody.

Covalent attachment of polypeptide or antigen binding agent to a solid support may generally be achieved by first reacting the support with a bifunctional reagent that will react with both the support and a functional group, such as a hydroxyl or amino group, on the polypeptide or antigen binding agent. For example, the polypeptide may be bound to supports having an appropriate polymer coating using benzoquinone or by condensation of an aldehyde group on the support with all amine and an active hydrogen on the polypeptide (see, e.g., Pierce Immunotechnology Catalog and Handbook, 1991, at A12-A13).

In certain embodiments, the assay is an enzyme immunoassay (EIA) or enzyme-linked immunosorbent assay (ELISA). In these assays, an enzyme, which is bound to the polypeptide or antigen-binding agent will react with an appropriate substrate, e.g., a chromogenic substrate, in such a manner as to produce a chemical moiety which can be detected, for example, by spectrophotometric, fluorimetric or by visual means. See e.g., Voller, A., “The Enzyme Linked ImmunoSorbent Assay (ELISA)” Microbiological Associates Quarterly Publication, Walkersville, Md., Diagnostic Horizons 2:1-7 (1978)); Voller et al., J. Clin. Pathol. 37:507-520 (1978); Butler, J. E., Meth. Enrymol. 73:482-523 (1981); Maggio, E. (ed.), Enzyme Immunoassay, CRC Press, Boca Raton, Fla., (1980); Ishikawa, E. et al., (eds.), Enzyme Immunoassay, Kgaku Shoin, Tokyo (1981).

In some embodiments, in an EIA or ELISA, the assay is performed by first contacting a polypeptide antigen that has been immobilized on a solid support, commonly the well of a microtiter plate, with a test sample, such that antibodies to the polypeptide within the sample are allowed to bind to the immobilized polypeptide. Unbound sample is then removed from the immobilized polypeptide and a detection reagent capable of binding to the immobilized antibody-polypeptide complex is added. The amount of detection reagent that remains bound to the solid support is then determined using a method appropriate for the specific detection reagent.

More specifically, once the polypeptide is immobilized on the support as described above, the remaining protein binding sites on the support are typically blocked. Any suitable blocking agent known to those of ordinary skill in the art, such as bovine serum albumin or Tween 20™ (Sigma Chemical Co., St. Louis, Mo., USA) may be employed. The immobilized polypeptide is then incubated with the sample, and antibody is allowed to bind to the polypeptide. The sample may be diluted with a suitable diluent, such as phosphate-buffered saline (PBS) prior to incubation. In some embodiments, an appropriate contact time (i.e., incubation time) is that period of time that is sufficient to detect the presence of antibody within a test biological sample. In some embodiments, the contact time is sufficient to achieve a level of binding that is at least 95% of that achieved at equilibrium between bound and unbound antibody. Those of ordinary skill in the art will recognize that the time necessary to achieve equilibrium may be readily determined by assaying the level of binding that occurs over a period of time. At room temperature, an incubation time of about 30 minutes is generally sufficient.

Unbound sample may then be removed by washing the solid support with an appropriate buffer, such as PBS containing 0.1% (v/v) Tween 20™. Detection reagent may then be added to the solid support. An appropriate detection reagent is any compound that binds to the immobilized antibody-polypeptide complex and that can be detected by any of a variety of means known to those in the art. Suitable detection reagents include, but are not limited to binding agents such as, Protein A, Protein C, immunoglobulin, lectin or free antigen conjugated to a reporter group. Suitable reported groups include, but are not limited to, e.g., enzymes (such as horseradish peroxidase and alkaline phosphatase), substrates, cofactors, inhibitors, dyes, radionuclides, luminescent groups, fluorescent groups, biotin and colloidal particles, such as colloidal gold and selenium. The conjugation of binding agent to reporter group may be achieved using standard methods known to those of ordinary skill in the art. Common binding agents may also be purchased conjugated to a variety of reporter groups from many commercial sources (e.g., Zymed Laboratories, San Francisco, Calif., USA, and Pierce, Rockford, Ill., USA).

Enzymes which can be used to detectably label the polypeptide-antibody complex formed include, but are not limited to, malate dehydrogenase, staphylococcal nuclease, delta-5-steroid isomerase, yeast alcohol dehydrogenase, alpha-glycerophosphate, dehydrogenase, triose phosphate isomerase, horseradish peroxidase, alkaline phosphatase, asparaginase, glucose oxidase, beta-galactosidase, ribonuclease, urease, catalase, glucose-6-phosphate dehydrogenase, glucoamylase and acetylcholinesterase. Additionally, the detection can be accomplished by colorimetric methods which employ a chromogenic substrate for the enzyme. Detection may also be accomplished by visual comparison of the extent of enzymatic reaction of a substrate in comparison with similarly prepared standards.

Detection may also be accomplished using fluorescence emitting metals such as ¹⁵²Eu, or others of the lanthanide series. These metals can be attached to the detection agent using such metal chelating groups as diethylenetriaminepentacetic acid (DTPA) or ethylenediaminetetraacetic acid (EDTA). Techniques for conjugating various moieties to antigen binding agent or polypeptide are well known see, e.g., Arnon et al., “Monoclonal Antibodies For Immunotargeting Of Drugs In Cancer Therapy”, in Monoclonal Antibodies And Cancer Therapy, Reisfeld et al. (eds.), Alan R. Liss, Inc. pp. 243-56 (1985); Hellstrom et al., “Antibodies For Drug Delivery”, in Controlled Drug Delivery (2nd Ed.), Robinson et al. (eds.), Marcel Dekker, Inc., pp. 623-53 (1987); Thorpe, “Antibody Carriers Of Cytotoxic Agents In Cancer Therapy: A Review”, in Monoclonal Antibodies '84: Biological And Clinical Applications, Pinchera et al. (eds.), pp. 475-506 (1985); “Analysis, Results, And Future Prospective Of The Therapeutic Use Of Radiolabeled Antibody In Cancer Therapy”, in Monoclonal Antibodies For Cancer Detection And Therapy, Baldwin et al. (eds.), Academic Press pp. 303-16 (1985), and Thorpe et at., “The Preparation And Cytotoxic Properties Of Antibody-Toxin Conjugates”, Immunol. Rev. (52:119-58 (1982).

The detection reagent is then incubated with the immobilized antibody-polypeptide complex for an amount of time sufficient to detect the bound antibody. An appropriate amount of time may generally be determined from the manufacturer's instructions or by assaying the level of binding that occurs over a period of time. Unbound detection reagent is then removed and bound detection reagent is detected using the reporter group. The method employed for detecting the reporter group depends upon the nature of the reporter group. For radioactive groups, scintillation counting or autoradiographic methods are generally appropriate. Spectroscopic methods may be used to detect dyes, luminescent groups and fluorescent groups. Biotin may be detected using avidin, coupled to a different reporter group (commonly a radioactive or fluorescent group or an enzyme). Enzyme reporter groups may generally be detected by the addition of substrate (generally for a specific period of time), followed by spectroscopic or other analysis of the reaction products.

All known variants of ELISA type assays may be used in the methods of the present technology, including but not limited to, e.g., indirect ELISA, sandwich EISA, competitive ELISA (see e.g., U.S. Pat. Nos. 5,908,781 and 7,393,843). Additionally other ELISA methods known in the art may be used in the methods of the present technology.

Other assays for use in the methods of the present technology include radioimmunoassay (RIA) (see, e.g., Weintraub, B., Principles of Radioimmunoassays, Seventh Training Course on Radioligand Assay Techniques, The Endocrine Society, (March, 1986)). The agent used to detect the polypeptide-antigen binding complex may be radioactively labeled. The radioactive isotope can be detected by means including, but not limited to, e.g., a gamma counter, a scintillation counter, or autoradiography.

Assays also include a rapid flow-through or strip test format, wherein the antigen is immobilized on a membrane, such as nitrocellulose. In the flow-through test, antibodies within the sample bind to the immobilized polypeptide as the sample passes through the membrane. A detection reagent (e.g., protein A-colloidal gold) then binds to the antibody-polypeptide complex as the solution containing the detection reagent flows through the membrane. The detection of bound detection reagent may then be performed. In the strip test format, one end of the membrane to which polypeptide is bound is immersed in a solution containing the sample. The sample migrates along the membrane through a region containing detection reagent and to the area of immobilized polypeptide. Concentration of detection reagent at the polypeptide indicates the presence of anti-Rv1168c antibodies in the sample. Typically, the concentration of detection reagent at that site generates a pattern, such as a line, that can be read visually. The absence of such a pattern indicates a negative result. In general, the amount of polypeptide immobilized on the membrane is selected to generate a visually discernible pattern when the test biological sample contains a level of antibodies that would be sufficient to generate a positive signal in an EISA, as discussed above. The amount of polypeptide immobilized on the membrane ranges from about 25 ng to about 1 μg, and from about 50 ng to about 500 ng. Such tests can typically be performed with a very small amount (e.g., one drop) of patient serum or blood.

To determine the presence or absence of M. tuberculosis Rv1168c polypeptides or antibodies in the test biological sample, the signal detected from the reporter group that remains bound to the solid support is generally compared to a signal that corresponds to a predetermined cut-off value. The cut-off value may be equivalent to the average mean signal obtained when the immobilized antigen is incubated with samples from an uninfected patient. Generally, a sample generating a signal that is about two standard deviations (SD), about three SD, about tour SD, about five SD, about six SD, about 7 SD, about 8 SD, about 9 SD and about 10 SD above the predetermined cut-off value is considered positive for M. tuberculosis. In an alternate method, the cut-off value is determined using a Receiver Operator Curve, according to the method of Sackett et al., clinical Epidemiology: A Basic Science for Clinical Medicine, Little Brown and Co., 1985, pp. 106-107. Briefly, in this method, the cut-off value may be determined from a plot of pairs of true positive rates (i.e., sensitivity) and false positive rates (100%-specificity) that correspond to each possible cut-off value for the diagnostic test result. The cut-off value on the plot that is the closest to the upper left-hand corner (i.e., the value that encloses the largest area) is the most accurate cut-off value, and a sample generating a signal that is higher than the cut-off value determined by this method may be considered positive or indicative of M. tuberculosis. Alternatively, the cutoff value may be shifted to the left along the plot, to minimize the false positive rate, or to the right, to minimize the false negative rate. In general, a test biological sample generating a signal that is higher than the cut-off value determined by these or any other methods known to one of skill in the art is considered indicative of exposure to or infection by M. Tuberculosis.

Additionally, the presence or absence of M. tuberculosis Rv1168c polypeptides or antibodies in the test biological sample may be determined by comparing the signal generated by the test biological sample in the assay to a historical cut-off value which was determined previously.

Additionally peripheral blood mononuclear cells (PBMCs) from patients or subjects requiring testing for M. tuberculosis exposure or infection may be used to detect or diagnose exposure to or infection by M. tuberculosis. Generally, the PBMCs from test subjects or mammals are isolated and then cultured. After several days of culturing, the Rv1168c polypeptide is added to the medium. After about 1-5 days, cell culture supernatants are collected and cytokine production by the PBMCs is measured by ELISA. In one embodiment, the amount of cytokine secreted by the PBMCs is compared to a control PBMC sample from a patient who has not been exposed to M. tuberculosis. A greater or lesser amount of cytokine present in the test sample is indicative of exposure to and/or infection by M. tuberculosis. Similar assays are described in more detail in Dillon, et al., J. of Clinical Microbiol. 38:3285-3290 (2000) and in U.S. Pat. No. 7,387,882.

Cytokines which can be measured in the PBMC assay described above, include but are not limited to any cytokine which the PBMCs can produce (e.g., IFN-γ, IL-5, and IL-2).

Of course, numerous other assay protocols exist that are suitable for use in the methods of the present technology. The above descriptions are intended to be illustrative only.

### Kits

In another aspect, kits are provided which contain at least one Rv1168c polypeptide, polypeptide binding agents fragments or variants thereof that can be prepared for detection, diagnosis, prognosis and/or monitoring M. tuberculosis exposure and/or infection by the assays described above. The components of the kits can be packaged either in aqueous medium or in lyophilized form. When the Rv1168c polypeptides, polypeptide binding agents fragments or variants thereof are used in the kits in the form of conjugates in which a label moiety is attached, such as an enzyme or a radioactive metal ion, the components of such conjugates can be supplied either in fully conjugated form, in the form of intermediates or as separate moieties to be conjugated by the user of the kit.

Additionally, the kit may comprise any other detection reagents (e.g., secondary labeled antibodies or enzyme substrate) needed for carrying out the assays described herein.

A kit may comprise a carrier being compartmentalized to receive in close confinement therein one or more container means or series of container means such as test tubes, vials, flasks, bottles, syringes, or the like. A first of said container means or series of container means may contain the Rv1168c polypeptides, polypeptide binding agents fragments or variants thereof. A second container or series of container means may contain a label or linker-label intermediate capable of binding to the primary antibody (or fragment thereof) or Rv1168c polypeptide or fragment thereof. Additionally, the kit will contain instructions for its use.

### EXAMPLES

The present technology is further illustrated by the following examples, which should not be construed as limiting in any way. The methods that were used in the experiments presented in this Section are described below.

### Example 1

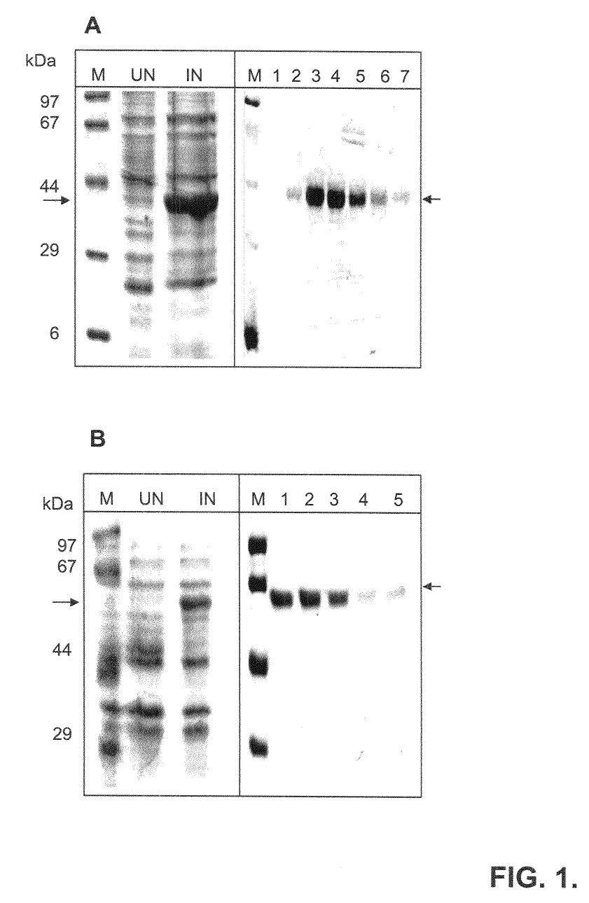

Cloning, expression and purification of recombinant Rv1168c and Hsp60 protein. The open reading frame corresponding to Rv1168c was PCR-amplified from the genomic DNA of M. tuberculosis H37Rv. XhoI and HindIII restriction sites were incorporated in the 5′ end of the forward and reverse primers respectively. The primers and the parameters for thermal cycle amplification are shown in Table 1. The PCR product was first directly cloned in the intermediate pGEM-T Easy vectors (Promega, Madison, ISA), followed by subcloning in bacterial expression vector pRSET-A (Invitrogen, Carlsbad, Calif.) in frame with a six N-terminal histidine tag using XhoI and HindIII. The clones were validated by sequencing with the T7 promoter primer on an Applied Biosystems Prism 377 DNA sequencer The pRSET-A clone was then transformed in BL21 (DE3)pLys expression system. The transformed cells were grown in Terrific Broth media containing ampicillin (100 μg/ml) and chloramphenicol (35 μg ml) and grown at 37° C. on a shaker to an OD₆₀₀ of 0.4-0.6, induced with 1 mM (isopropyl-β-D-thiogalactopyranoside (IPTG), and further grown at 37° C. for 3-4 hours. The cells were lysed and induction of Rv1168c was checked (FIG. 1A).

TABLE 1
PCR Primers and thermal cycle conditions for amplification of Rv1168c

| | | | |
|---|---|---|---|
| | | | AMPLICON |
| PRIMER | SEQUENCE | PCR CONDITIONS | SIZE |

| | | | | |
|---|---|---|---|---|
| Forward | gactcgagatggatttcacaattttt | 94° C. for 12 min. | | ~1041 bp |
| | (SEQ ID NO: 3) | | | |
| Reverse | gcaagcttctagccggcggcgggtgaccgcagt | 94° C. for 30 sec | 10 cycles | |
| | (SEQ ID NO: 4) | 42° C. for 30 sec. | | |
| | | 72° C. for 1 min. | | |
| | | 94° C. for 30 sec. | 20 cycles | |
| | | 37° C. for 30 sec. | | |
| | | 72° C. for 1 min. | | |
| | | 72° C. for 30 min. | | |

Polyhistidine tagged recombinant protein was purified using TALON resin (BD Biosciences Clontech) according to manufacturer's recommendation for purification of protein under native conditions. The purity of the protein was confirmed by loading onto a 10% SDS gel, A single protein band with molecular weight of ˜42 kDa corresponding to the Rv1168c protein was observed. The yield of protein was 6 mg/L culture and appeared to be 98% pure (FIG. 1A). The mycobacterial heat shock protein 60 (Hsp60) was purified (FIG. 1B) as described (30). The purified recombinant ESAT-6 protein of Mtb was a gift of Dr. Pawan Sharma, ICCEB, New Delhi, India. Protein concentrations were estimated using the bicinchonic acid method (Micro BCA Protein Assay Kit; Pierce, Rockford, USA), To remove endotoxin contamination, purified Rv1168c or Hsp60 or ESAT-6 protein was incubated with 10% (v/v) polymyxin B-agarose (Sigma-Aldrich; binding capacity, 200 to 500 μg of LPS from Escherichia coli serotype O128: B12/ml) for 1 hour at 4° C. and the protein preparation was used to assess the B-cell or the T-cell responses.

Study population. The study population (n=109) was comprised of pulmonary (n=77) and extrapulmonary (n=32) tuberculosis patients diagnosed at the DOTS (Directly Observed Treatment−Short-course) Clinic of Mahavir Hospital and Research Centre, Hyderabad, India. The diagnosis of the patients with pulmonary TB was based on the results of sputum smear for the presence of acid-fast bacillus, radiographic examination and clinical symptoms as per the RNTCP (Revised National TB Control Programme, Central Division, Directorate General of Health Services, Ministry of Health and Family Welfare, Government of India, www.tbcindia.org) guidelines. The extrapulmonary cases were confirmed by tissue biopsy, clinical symptoms and radiographic evidence (www tbcindia.org). All the subjects were found to be negative for HIV. Sera were collected from the patients just before initiation of chemotherapeutic regime. All the patients responded to the DOTS regime and subsequently, the patients were considered cured based on relief from clinical symptoms, absence of the acid-fast bacillus in the sputum, and radiographic examination. Control sera (n=20) were collected from volunteers from TB endemic regions. All the control subjects were BCG-vaccinated and had no clinical symptoms of TB at the time of sample collection. The bioethics committee of Mahavir Hospital and research Centre and CDFD approved the present study, and written and informed consent was obtained from all of the subjects.

Enzyme immunoassay (EIA), For EIA, 96-well microtiter plates (Costar, Corning, N.Y.) were coated with 0.5 μg/well recombinant Rv1168c, ESAT-6, Hsp60 protein or PPD (diluted in 0.1M carbonate buffer, pH 9.5 and 50 μl was added to each well) (10). Plates were incubated overnight at 4° C., washed three times with phosphate buffer saline (PBS) and blocked with 100 μl of blocking buffer (2% (w/v) BSA in PBS) for 2 hours at 37° C. After washing the plates three times with PBS containing 005% (v/v) Tween-20 (Sigma-Aldrich) (PBS-T) sera (200 times diluted in blocking buffer) from various study groups were added (50 μl/well) to antigen-coated wells in duplicate and incubated for 1 hour at 37° C. The plates were washed for 3 times with PBS-T and incubated with 50 μl/well of anti-human immunoglobulin G (IgG)-horseradish peroxidase (HRP) (Sigma-Aldrich) conjugate (1:8000 dilution in blocking buffer) at 37° C. for 1 hour. The plates were washed for 2 times with PBS-T and a final wash was carried out with PBS. The HRP activity was detected using a chromogenic substance, o-phenylenediamine tetrahydrochloride (Sigma-Aldrich) in citrate-phosphate buffer (pH 5.4) and H₂O₂ (Merck, Germany) as substrate (1 μl/ml). Reactions were terminated using 1N H₂SO₄ and the absorbance values were measured at 492 nm in an EIA reader (Bio-Tek Instruments Inc., Vermont, USA).

Cytokine assay. The peripheral blood mononuclear cells (PBMCs) from TB patients (n=35) and BCG-vaccinated controls (n=10) were isolated using density gradient centrifugation in Ficoll-Hypaque (Sigma-Aldrich) solution as described elsewhere (9), and prepared at 2.5×10⁶ cells/ml in RPMI-1640 (Invitrogen, Grand Island, N.Y., USA) medium containing 10% fetal bovine serum (FBS; Invitrogen) and antibiotics (RPMI-10). Cell suspensions (200 μl/well) were dispensed into 96-well, flat-bottom microtiter plates (Nunc, Roskilde, Denmark) and maintained at 37° C. in 5% CO₂ incubator. PBMCs from various groups were treated with a fixed concentration of Rv1168c (3 μg/ml) or PPD aid after 4 days culture supernatants were harvested for estimating interferon-gamma (IFN-γ) and interleukin-5 (IL-5) cytokines, secreted in the culture supernatants, by EIA, The cytokine was quantified by two-site sandwich EIA (BD Biosciences Pharmingen, San Diego, Calif.) following the manufacturer's protocol as described (24). Briefly, 96-well polyvinyl chloride microtiter plates were coated with purified anti-cytokine antibody at 2 μg/ml concentration. The plates were blocked with 2% BSA in PBS and incubated with various culture supernatants followed by incubation with biotin conjugated anti-cytokine antibody and streptavidin-HRP. The HRP activity was detected using o-phenylenediamine tetrahydrochloride and absorbance was read at 492 nm. Standard curve for the cytokine was obtained using the IFN-γ or IL-5 recombinant standard protein provided by the manufacturer.

Statistical analysis. For evaluation of antibody responses, cutoff values were calculated for each antigen as the means of OD₄₉₂ values obtained with the sera from 20 healthy donors (BCG-vaccinated controls) plus 6 standard deviation (SD) (27, 28). Data were analyzed following student's t test or ANOVA as indicated. P<0.05 was considered to be significant.

### Example 2

### The Rv1168c Polypeptide Shows a Strong Immunoreactivity Towards TB Patient Sera Compared to that of BCG-Vaccinated Controls

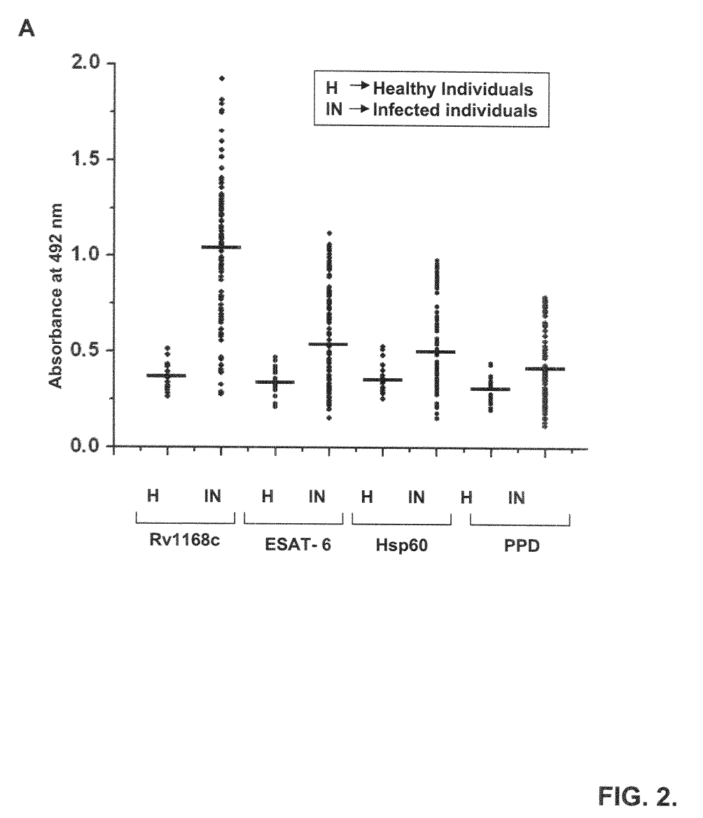

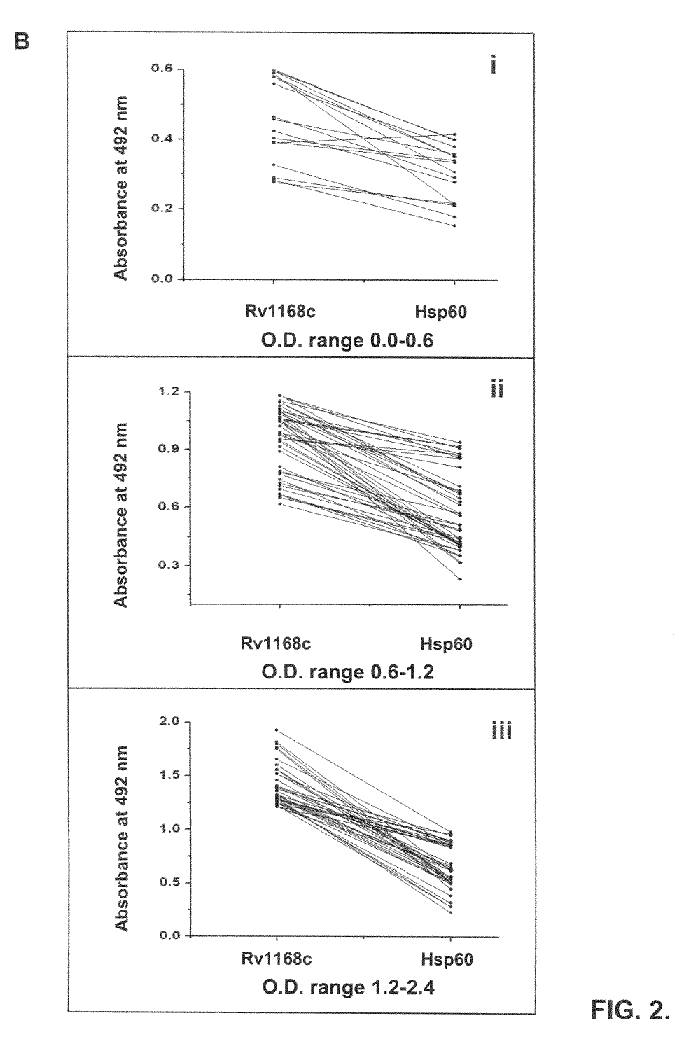

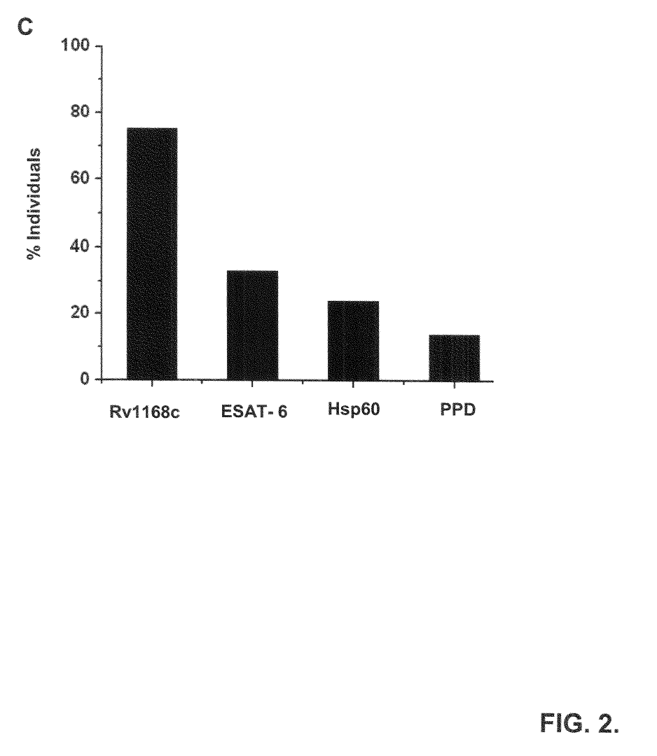

Based on its predominant expression during the conditions that mimic in vivo phagosomal environment (2, 5, 31, 35), and high antigenicity index, it was hypothesized that the Rv1168c polypeptide may induce a strong B-cell response in people having active TB infection. In the study described herein the immunological potential of the Rv1168c polypeptide as a diagnostic marker in a cohort of clinically defined active TB patients and BCG-vaccinated controls was evaluated. The specific antibody reactivity in response to Rv1168c protein in sera from TB patients compared to BCG vaccinated controls was examined. The antibody titers (FIG. 2A) against Rv1168c were found to be significantly higher (mean absorbance value at 492 nm [OD₄₉₂]±SD=1.05±0.381) in TB patients compared to that of the BCG-vaccinated control sera (OD₄₉₂±SD=0.373±0.066; P<0.0001).

The sensitivity and specificity of Rv1168c immunoreactivity was compared with the responses elicited by ESAT-6 (7, 12) and Hsp60 (3) and PPD. The levels of anti-PPD antibodies were found to be low in TB patients (OD₄₉₂±SD was 0.415±0.184) indicating that PPD was not very sensitive in detecting patients with active TB, which was in agreement with other reports (10, 28). Although ESAT-6 (OD₄₉₂±SD was 0.612±0.264) was a better antigen than PPD, the sera of the patients reacted very strongly against Rv1168c than ESAT-6 (FIG. 2A). Similarly, although the Hsp60 protein showed a better response (OD₄₉₂±SD was 0.571±0.230) than PPD, it still had a lower reactivity than the Rv1168c polypeptide in a majority of the TB patients (FIG. 2B). The sera of the patients reacted very strongly against Rv1168c as compared to ESAT-6, Hsp60 and PPD (P<0.0001 in all cases), Therefore, the Rv1168c protein is more efficient in discriminating active tuberculosis patients from the BCG-vaccinated controls compared to Hsp60 and PPD. When the proportion of highly reactive sera (antibody levels greater than or equal to the mean OD₄₉₂ of BCG-vaccinated control sera plus 6 SD) among responders to each antigen was calculated, it was observed that Rv1168c elicited high level antibody responses in the majority (75.2%) of responders as compared to PPD (14%) and Hsp60 (24%) and ESAT-6 (33.1%) (FIG. 2C). Thus, it appears that the Rv1168c polypeptide is more immunodominant and serologically more sensitive than PPD, Hsp60 and ESAT-6.

Thus, the data demonstrate that as compared to the conventional diagnostic test using PPD, recombinant Rv1169c is highly sensitive to distinguish patients with active tuberculosis from BCG-vaccinated controls. In addition, recombinant Rv1168c was found to be more sensitive than ESAT-6 and Hsp60 (well-known immunodominant antigens of Mtb) in recognizing TB patients from BCG-vaccinated controls. Interestingly, although a homologue of Rv1168c is present in M. bovis, a negligible immunological response to this protein was found in BCG-vaccinated individuals indicating that Rv1168c is probably highly expressed during the active pathogenesis of M. tuberculosis. These results suggest that Rv1168c antigen may be considered as an attractive candidate for development of new diagnostic tests that can identify people suffering from the active form of the disease in TB endemic regions.

### Example 3

### The Rv1168c Polypeptide can Detect Patients with Extrapulmonary and Smear-Negative TB Serologically

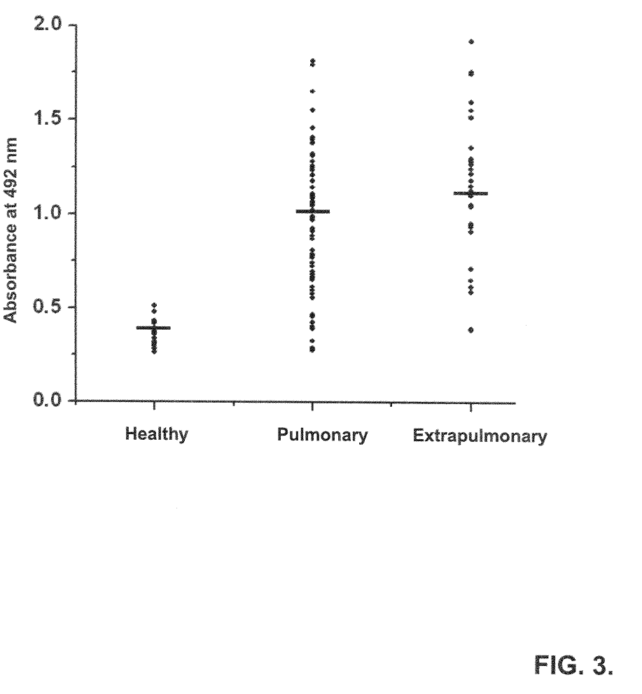

Since the recombinant Rv1168c protein was found to be sero-reactive against most of the TB patients, the antibody titers specific to Rv1168c in well-defined clinical categories like pulmonary and extrapulmonary cases were compared. Due to limitations in the current array of diagnostic methods, diagnosis of extrapulmonary cases (which are mostly sputum negative) is more difficult than diagnosis of pulmonary TB. Therefore, a diagnostic method with potential to identify patients with extrapulmonary TB would be highly valuable. The Rv1168c polypeptide elicited stronger antibody responses in extrapulmonary patients in addition to the pulmonary cases as compared to BCG-vaccinated controls (FIG. 3; P<0.0001 in both cases). The mean absorbance value for Rv1168c in control, pulmonary and extrapulmonary groups were 0.373, 1.01 and 1.15 respectively (FIG. 3). When the immunogenicity of Rv1168c over ESAT-6, Hsp60 and PPD was compared, the data presented in Table 2 clearly show that the mean reactivity of Rv1168c was significantly higher in comparison to that of ESAT-6 (P<0.0001), Hsp60 (P<0.0001) and PPD (P<0.0001) in both pulmonary and extrapulmonary patient sera. When expressed as percentages of high-level responders showing antibody levels greater than or equal to cutoff values (mean OD₄₉₂ of BCC-vaccinated control sera plus 6 SD), the majority of the pulmonary (73%) and extrapulmonary (81.3%) individuals showed antibody levels greater than the cutoff value against Rv1168c antigen whereas only 37.6% and 21.9% responders had higher levels against ESAT-6, 27.2% and 16% against Hsp60 and 14.3% and 12.5% against PPD respectively (Table 2).

TABLE 2
The Rv1168c protein can detect smear-positive and smear-negative pulmonary
as well as extrapulmonary TB cases*

| | | | | | Smear-positive | | Smear-negative | |
|---|---|---|---|---|---|---|---|---|
| | Total Extrapulmonary | | Total Pulmonary | | ulmonary | | pulmonary | |
| | cases (n = 32) | | cases (n = 77) | | cases (n = 53) | | cases (n = 24) | |
| | | % | | % | | % | | % |
| Antigen | Mean ± SD | Responders | Mean ± SD | Responders | Mean ± SD | Responders | Mean ± SD | Responders |
| Rv1168c | 1.15 ± 0.38 | 81.3 | 1.01 ± 0.38 | 73.0 | 1.01 ± 0.36 | 71.6 | 1.03 ± 0.42 | 75.0 |
| ESAT-6 | 0.62 ± 0.22 | 21.9 | 0.61 ± 0.28 | 37.6 | 0.60 ± 0.26 | 34.0 | 1.64 ± 0.32 | 45.8 |
| Hsp60 | 0.52 ± 0.22 | 16.0 | 0.59 ± 0.23 | 27.2 | 0.61 ± 0.22 | 28.3 | 0.56 ± 0.24 | 25.0 |
| PPD | 0.41 ± 0.17 | 12.5 | 0.42 ± 0.19 | 14.3 | 0.40 ± 0.16 | 9.4 | 0.46 ± 0.23 | 21.0 |

*The data values in FIG. 2A were replotted to compare the antibody response of the pulmonary and extrapulmonary TB patients against Rv1168c versus ESAT-60, Hsp60 and PPD. Percentage of responders showing the absorbance value greater than or equal to the cutoff value (mean OD₄₉₂ plus 6 SD, obtained with BCG-vaccinated control sera) was compared for pulmonary and extrapulmonary groups. The pulmonary TB cases were further categorized as smear-positive and smear-negative and responder to Rv1168c was compared with that of ESAT-6, Hsp60 and PPD by calculating the percentage of individuals showing absorbance values greater than or equal to mean OD₄₉₂ plus 6 SD, obtained with vaccinated control sera.

Like the extrapulmonary cases, the smear-negative pulmonary TB cases are also difficult to detect. Anti-Rv1168c antibody titers were examined to determine if they were higher in the smear-negative pulmonary TB. Interestingly Rv1168c was more sensitive than the ESAT-6, Hsp60 and PPD in detecting smear-negative pulmonary TB patients. It was found that in smear-negative TB patients (n=24), serum samples from 75% of the patients had antibodies to Rv1168c whereas only 45.8% of patients had antibodies to ESAT-6, 25% of patients had antibodies to Hsp60 and 21% of patients had antibodies to PPD (Table 2). In the cohort of smear-positive TB patients (n=53), 71.6% possessed Rv1168c specific, 34% had ESAT-6, 28.3% had Hsp60 specific, and 9.4% had PPD specific antibodies (Table 2). These results indicate that Rv1168c can detect all the categories of TB patients such as the smear-negative pulmonary, smear-positive pulmonary and extrapulmonary TB cases with higher sensitivity as compared to ESAT-6, Hsp60 and PPD.

Despite the initial clinical suspicion of TB, when a patient's sputum smear results are negative for acid-fast bacilli, the diagnosis of TB may be missed. Therefore, it is important to continue research for rapid and reliable immunological tests to diagnose smear-negative TB cases (23, 37). Recent approaches, using ESAT-6 and CFP-10 as diagnostic antigens, are useful mostly either to detect the latent infection or the sputum positive pulmonary patients and not much information is available to use these antigens to diagnose the smear-negative cases with higher sensitivity (14, 23, 37). The Rv1168c polypeptide can detect the extrapulmonary and the smear-negative pulmonary TB cases with higher sensitivity as compared to ESAT-6 as well as Hsp60 immunodominant antigens. A very high percentage of the serum samples obtained from the extrapulmonary and the smear-negative pulmonary TB patients had strong antibody reactivities against the Rv1168c protein as compared to ESAT-6, Hsp60 and PPD indicating that Rv1168c can be used to detect these categories of TB patients with higher sensitivity and can discriminate smear-negative pulmonary as well as extrapulmonary patients from the BCG-vaccinated controls. Thus, these findings are particularly significant in the context of smear-negative pulmonary and extrapulmonary TB cases which often go undetected with conventional diagnostic methods (4, 8).

### Example 4

### The PPE Antigen Rv1168c Mounts a Strong T-Cell Response in TB Patients as Compared to a BCG-Vaccinated Control Group

In Vitro tests measuring IFN-γ production by whole blood cells have been discussed to detect active TB cases (14, 23). The Rv1168c polypeptide mounted stronger antibody responses of IgG type in patients with active TB (FIGS. 2 and 3, Table 2). IgG isotype switching requires a direct interaction from T-cells (14). Therefore, there is a possibility that the Rv1168c polypeptide can also induce stronger T-cell response in TB patients. Therefore, T-cell responses, indicated by the amount of cytokines secreted in vitro in TB patients (with pulmonary tuberculosis and extrapulmonary tuberculosis) and BCG-vaccinated controls, were measured. IFN-γ levels in both pulmonary (mean=287 pg/ml) and extrapulmonary (mean=325 pg/ml) TB patients were elevated to more than 3-fold when compared with the levels (mean=92 pg/ml) in BCG-vaccinated controls (Fable 3; P<0.0001 in both cases). Similarly, IL-5 levels were also significantly elevated (Table 3; P<0.0001) in both pulmonary (mean=147 pg ml) and extrapulmonary (mean=191 pg/ml) TB patients when compared with BCG-vaccinated controls (mean=80 pg/ml). Also, it was observed that as compared to PPD, the Rv1168c polypeptide is a potent T-cell antigen in detecting active TB cases (Table 3). These data indicate that T-cells from TB patients respond dominantly against the mycobacterial Rv1168c protein and can distinguish TB patients from BCG vaccinated controls.

TABLE 3
The Rv1168c protein mounts stronger T-cell responses in TB patients
as compared to BCG-vaccinated individuals*

| | Healthy | | Pulmonary | | Extrapulmonary | |
|---|---|---|---|---|---|---|
| | IFN-γ | IL-5 | IFN-γ | IL-5 | IFN-γ | IL-5 |
| Antigen | (pg/ml) | (pg/ml) | (pg/ml) | (pg/ml) | (pg/ml) | (pg/ml) |
| Rv1168c | 92 ± 24 | 80 ± 22 | 287 ± 70 | 147 ± | 325 ± 125 | 191 ± |
| | | | | 69 | | 61 |
| PPD | 91 ± 17 | 78 ± 19 | 177 ± 52 | 81 ± | 141 ± 51 | 106 ± |
| | | | | 31 | | 50 |

*PMBCs collected from TB patients and BCG-vaccinated controls were stimulated in triplicate with 3 μg/ml of Rv1168c or 10 μg/ml PPD. After 4 days levels of IFN-γ and IL-5 secreted in the culture supernatants were estimated by EIA. Statistical analysis was performed using Students t-test. Data are expressed as the mean ± SD.

The generation of substantive antibody responses to a protein antigen is dependent on the presence of T-cell epitopes recognized by helper T cells (14). As disclosed herein, it was observed that the Rv1168c polypeptide is also a potent T-cell antigen, eliciting higher levels of IFN-γ in the PBMCs obtained from TB patients in contrast to those obtained from the BCG-immunized controls. Thus, Rv1168c is also a dominant T-cell antigen recognized by most of the TB patients and thus suggesting that the Rv1168c polypeptide plays an important role in certain stages of mycobacterial infection and intracellular survival.

### Example 5

### Rv1168c is more Potent in Detection of Pulmonary and Extrapulmonary TB Cases When Compared to Other PPE Proteins

A few PPE proteins have been studied to determine their suitability in serological diagnosis of TB patients. Rv1168c is more potent in the detection of both pulmonary and extrapulmonary TB cases when compared to some of the earlier studied PPE proteins such as Rv3425 (40), Rv2608 (9) and Rv2430 (10) in EIA. Rv1168c shows comparable immunogenicity to only Rv3872 (28), however in the present studies a more stringent cutoff was used. In the experiments previously described, a cutoff of OD plus 6 SD was used to discriminate patient sera from BCG-vaccinated control sera and still a significantly higher percentage of TB patients were detected (in both pulmonary and extrapulmonary TB) by Rv1168c as compared to the above mentioned PPE proteins. In previous studies with the PPE proteins mentioned above, calculations were made using a less stringent cutoff value of OD plus 3 SD, This suggests that the Rv1168c protein is practically more sensitive to distinguish patient sera from the BCG-immunized control sera. Rv1168c can detect almost 75% of the smear-negative cases effectively.

### Example 6

### Creation of Antigenic Fragments of Rv1168c Polypeptide

Antigenic fragments of the Rv1168c polypeptide can be used in all methods described herein. Specifically, antigenic fragments of Rv1168c are identified using, for example, computer modeling programs that analyze the antigenic profile of polypeptides. Such programs include, but are not limited to, e.g., DNAStar, DS Gene, PEOPLE, CEP, BEPITOPE, and PREDITOP. Additionally, antigenic regions of a protein are predicted using hydropathy plots that assign an average hydrophilicity and hydrophobicity for each amino acid residue in a sequence. The highest point of average hydrophilicity for a series of contiguous amino acids is an indicator of a region of the polypeptide that is exposed and, thus, a potential antigenic region. Non-limiting examples of hydropathy plots include methods developed by Kyte and Doolittle; Jameson and Wolf, and Cliou and Fasman (41, 42, 43).

Table 4 below lists predicted antigenic regions of Rv1168c (SEQ ID NO: 1) as determined by the method of Kyte and Doolittle (41):

TABLE 4
Predicted Antigenic Regions of Rv1168c

| | |
|---|---|
| Amino Acid Positions in SEQ ID NO:1 | Sequence |

| | |
|---|---|
| 10-18 | FNSLNIQGS |
| | (SEQ ID NO: 5) |
| 29-35 | WKNLSNE |
| | (SEQ ID NO: 6) |
| 37-46 | SYAASRFESE |
| | (SEQ ID NO: 7) |
| 52-59 | TSWRGPSS |
| | (SEQ ID NO: 8) |
| 109-114 | TNRLTL |
| | (SEQ ID NO: 9) |
| 138-149 | AQYWSQDGEAMN |
| | (SEQ ID NO: 10) |
| 196-203 | PATKNLPS |
| | (SEQ ID NO: 11) |
| 213-216 | TYPL |
| | (SEQ ID NO: 12) |
| 236-241 | SNMGQM |
| | (SEQ ID NO: 13) |
| 251-256 | TLTLKG |
| | (SEQ ID NO: 14) |
| 288-292 | WVADK |
| | (SEQ ID NO: 15) |
| 305-314 | APGGPSGTSW |
| | (SEQ ID NO: 16) |

Antigenic fragments of Rv1168c are generated by methods known in the art. Non-limiting examples include recombinant methods such as the generation of Rv1168c polynucleotide fragments by the polymerase chain method (PCR) and subsequent cloning of the DNA fragments into an expression vector for expression of the fragments in a mammalian expression system. Additionally, the polypeptide fragments described above, or any other fragments of Rv1168c, can be chemically synthesized. Select methods useful for the chemical synthesis as well as other methods for preparing fragments of Rv1168c are described herein.

The antigenic fragments listed above, as well as any other fragments of Rv1168c, are then tested in any of the assays described herein to determine their utility as diagnostic agents for detecting and diagnosing exposure or infection to M. tuberculosis. Non-limiting examples of assays which can be used to test the antigenic properties of Rv1168c polypeptide fragments, as well as their use in the assays described herein, include, but are not limited to, e.g., Western immunoblotting with Rv1168c polypeptide fragments and serum samples from M. tuberculosis positive patients or purified anti-Rv1168c antibodies, and ELISA assays. Additionally, Rv1168c fragments are tested for their antigenic properties and usefulness as diagnostic markers following the methods described in Li et al. (44).

### REFERENCES

1. Abdallah, A. M., T. Verboom, F. Hannes, M. Safi, M. Strong, D. Eisenberg, R. J. Musters, C. M. Vandenbroucke-Grauls, B. J. Appelmelk, J. Luirink, and W. Bitter. 2006. A specific secretion system mediates PPE41 transport in pathogenic mycobacteria. Mol. Microbiol. 62:667-679.
2. Bacon, J. B. W. James, L. Wernisch, A, Williams, K. A. Morley, G. J. Hatch, J. A, Mangan, J. Hinds, N. G. Stoker, P. D. Butcher, and P. D. Marsh. 2004. The influence of reduced oxygen availability on pathogenicity and gene expression in Mycobacterium tuberculosis. Tuberculosis 84:205-217.
3. Banerjee, S., A Nandyala, R. Podili, V. M. Katoch, K. J. Murthy, and S. E. Hasnain, 2004. Mycobacterium tuberculosis (Mtb) isocitrate dehydrogenases show strong B cell response and distinguish vaccinated controls from TB patients, 2004. Proc. Natl. Acad. Sci. USA 101: 12652-12657.
4. Barnes, P. F. 1997. Rapid diagnostic tests for tuberculosis: progress but no gold standard. Am. J. Respir. Crit. Care Med. 155:1497-1498.
5. Betts, J. C., P. T. Lukey, L. C. Robb, R, A, McAdam, and K. Duncan, 2002. Evaluation of a nutrient starvation model of Mycobacterium tuberculosis persistence by gene and protein expression profiling. Mol. Microbiol, 43:717-731.
6. Bock, N. N., J. E. McGowan, Jr, J, Ahn. J. Tapia, and H. M. Blumberg, 1996, Clinical predictors of tuberculosis as a guide for a respiratory isolation policy. Am. J. Respir. Crit. Care Med. 154:1468-1472.
7. Brock, I., M. E. Munk, A, Kok-Jensen and P. Andersen, 2001. Performance of whole blood IFN-gamma test for tuberculosis diagnosis based on PPD or the specific antigens ESAT-6 and CFP-10. Int. J. Tuberc. Lung Dis. 5:462-467.
8. Brugiere, O., M. Vokurka, D. Lecossier, G. Mangiapan. A. Amrane, B. Milleron, C. Mayaud, J. Cadranel, and A. J. Hance, 1997, Diagnosis of smear-negative pulmonary tuberculosis using sequence capture polymerase chain reaction. Am. J. Respir. Crit. Care Med. 155:1478-1481.
9. Chakhaiyar, P., Y. Nagalakshmi, B. Aruna, K. J. Murthy, V. M. Katoch, and S. E. Hasnain. 2004. Regions of high antigenicity within the hypothetical PPE major polymorphic tandem repeat open-reading frame, Rv2608, show a differential humoral response and a low T cell response in various categories of patients with tuberculosis. J. Infect. Dis. 190:1237-1244.
10. Choudhary R. K., S. Mukhopadhyay, P. Chakhaiyar, N. Sharma, K. J. Murthy, V. M. Katoch, and S. E. Hasnain, 2003, PPE antigen Rv2430c of Mycobacterium tuberculosis induces a strong B-cell response, Infect. Immun. 71:6338-6343.
11. Cole, S. T. 2002. Comparative and functional genomics of the Mycobacterium tuberculosis complex. Microbiology 148:2919-2928,
12. Demangel, C. P. Brodin, P. J. Cockle, R. Brosch, L., Majlessi. C. Leclerc, and S. T. Cole. 2004 Cell envelope protein PPE68 contributes to Mycobacterium tuberculosis RD1 immunogenicity independently of a 10-kilodalton culture filtrate protein and ESAT-6. Infect. Immun. 72:2170-2176.
13. Devi, K. R., K. S. Kumar, B. Ramalingam, and Alamelu R. 2002. Purification and characterization of three immunodominant proteins (38, 30, and 16 kDa) of Mycobacterium tuberculosis. Protein Expr. Purif. 24:188-195.
14. Dillon, D. C., M. R. Alderson, C. H. Day, T. Bement, A. Campos-Neto, Y. A. Skeiky, T. Vedvick, R. Badaro, S. G. Reed, and R. Houghton. 2000. Molecular and immunological characterization of Mycobacterium tuberculosis CFP-10, an immunodiagnostic antigen missing in Mycobacterium bovis BCG. J. Clin. Microbiol. 38:3285-3290.
15. Dillon, D. C., M. R. Alderson, C. H. Day, D. M. Lewinsohn, R. Coler, T. Bement. A, Campos-Neto, Y. A. Skeiky, I. M. Orme, A. Roberts, S. Steen, W. Dalemans, R. Badaro, and S. G. Reed. 1999 Molecular characterization and human T-cell responses to a member of a novel Mycobacterium tuberculosis mtb39 gene family. Infect. Immun. 67:2941-2950.
16. Dye, C., M. A. Espinal, C. J. Watt, C. Mbiaga, and B. G. Williams. 2002. Worldwide incidence of multidrug-resistant tuberculosis. J. Infect. Dis. 2002; 185:1197-1202.
17. Edwards, L. B., F. A. Acquaviva, V. T. Livesay, F. W. Cross, and C. E. Palmer. 1969. An atlas of sensitivity to tuberculin, PPD-B, and histoplasmin in the United States. Am. Rev. Respir. Dis. 99:1-132.
18. Friedland, G. 2007. Tuberculosis, Drug Resistance, and HIV/AIDS: A Triple Threat. Curr. Infect. Dis. Rep. 9:252-261.
19 Gey van Pittius N. C., S. L. Sampson, H. Lee, Y. Kim, P. D. van Helden, and R. M. Warren. 2006. Evolution and expansion of the Mycobacterium tuberculosis PE and PPE multigene families and their association with the duplication of the ESAT-6 (esx) gene cluster regions. BMC Evol. Biol. 6:95.
20. Harboe, M. 1981. Antigens of PPD, old tuberculin, and autoclaved Mycobacterium bovis BCG studied by crossed immunoelectrophoresis. Am. Rev. Respir. Dis. 124:80-87.
21. Houghton, R. L., M. J. Lodes, D. C. Dillon, L. D. Reynolds, C. H. Day, P. D. McNeill, R. C. Hendrickson, Y. A. Skeiky, D. P. Sampaio, R. Badaro, K. P. Lyashchenko, and S. G. Reed. 2002. Use of multiepitope polyproteins in serodiagnosis of active tuberculosis. Clin. Diagn. Lab. Immunol. 9:883-891.
22. Huebner, R. E., M. F. Schein, Jr. J. B. Bass, R. E. Huebner, M. F. Schein, and Jr. J. B. Bass. 1993. The tuberculin skin test. Clin. Infect, Dis. 17:968-975.
23. Jafari, C., M. Ernst, B. Kalsdorf, U. Greinert, R. Diel, D. Kirsten, K. Marienfeld, A. Lalvani, and C. Lange. 2006. Rapid diagnosis of smear-negative tuberculosis bybronchoalveolar lavage enzyme-linked immunospot. Am. J. Respir. Crit. Care Med. 429 174:1048-1054.
24. Khan, N., S. S. Rahim, C. S. Boddupalli, S. Ghousunnissa, S. Padma, N. Pathak, D. Thiagarajan, S. E. Hasnain, and S. Mukhopadhyay. 2006. Hydrogen peroxide inhibits IL-12 p40 induction in macrophages by inhibiting c-rel translocation to the nucleus through activation of calmodulin protein. Blood 107:1513-1520.
25. Lalvani, A., A. A. Pathan, H. McShane, R. J. Wilkinson, M. Latif, C. P. Conlon, G Pasvol, and A. V. Hill. 2001. Rapid detection of Mycobacterium tuberculosis infection by enumeration of antigen-specific T cells. Am. J. Respir. Crit. Care Med. 163: 824-828.
26. Ljungqvist, L., A. B. Andersen, P. Andersen, K. Hasløv, A. Worsaae, J. Bennedsen, and I. Heron. 1990, Affinity purification, biological characterization and serological evaluation of defined antigens from Mycobacterium tuberculosis. Trop. Med. Parasitol. 41:333-335.
27. Lyashchenko, K., R. Colangeli, M. Houde, H. Al Jahdali, D. Menzies, and M. L. Gennaro. 1998. Heterogeneous antibody responses in tuberculosis. Infect. Immun. 66:3936-3940.
28. Mukherjee, P., M. Dutta, P. Datta, R. Pradhan, M. Pradhan, M. Kund, J. Basu, and P. Chakrabarti. 2007. The RD1-encoded antigen Rv3872 of Mycobacterium tuberculosis as a potential candidate for serodiagnosis of tuberculosis Clin. Microbiol, Infect, 13:146-152.
29. Murphy, G. D., R. R. Rydman, D. Batesky, and S. Hill 1995. Clinical parameters that predict culture-positive pulmonary tuberculosis in the emergency department [abstract]. Ann. Emerg. Med. 25:137.
30. Mustafa, A. S. 2002. Development of new vaccines and diagnostic reagents against tuberculosis. Mol. Immunol. 2002; 39:113-119.
31. Muttucumaru, D. G., G. Roberts J. Hinds, R. A. Stabler, and T. Parish. 2004. Gene expression profile of Mycobacterium tuberculosis in a non-replicating state. Tuberculosis (Edinb) 84:239-246.
32, Okkels, L. M., I. Block, F. Follmann, E. M. Agger, S. M. Arend, T. H. Ottenhoff, F. Oftung, I. Rosenkrands, and P. Andersen. 2003. PPE protein (Rv3873) from DNA segment RD1 of Mycobacterium tuberculosis: strong recognition of both specific T-cell epitopes and epitopes conserved within the PPE family. Infect. Immun. 71:6116-6123.
33. Perkins, M. D. 2000. New diagnostic tools for tuberculosis. Int. J. Tuberc. Lung Dis. 4:S182-S188.
34. Qamra, R., V. Srinivas, and S. C. Mande. 2004. Mycobacterium tuberculosis GroEL homologues unusually exist as lower oligomers and retain the ability to suppress aggregation of substrate proteins. J. Mol. Biol. 342:605-617.
35, Schnappinger, D., S. Ehrt, M. I. Voskuil, Y. Liu, J. A. Mangan, I. M. Monahan, G. Dolganov, B. Efron, P. D. Butcher, C. Nathan, and G. K. Schoolnik. 2003. Transcriptional adaptation of Mycobacterium tuberculosis within macrophages: Insights into the phagosomal environment. J. Exp. Med. 198:693-704.
36. Singh, K. K., Y. Dong, S. A. Patibandla, D. N. McMurray, V. K. Arora, and S. Laal. 2005 Immunogenicity of the Mycobacterium tuberculosis PPE55 (Rv3347c) protein during incipient and clinical tuberculosis. Infect Immun. 73:5004-5014.
37. Streitz, M., L. Tesfa, V. Yildirim, A. Yahyazadeh, T. Ulrichs, R. Lenkei, A Quassem, G. Liebetrau, L. Nomura, H. Maecker, H. D. Volk, and F. Kern 2007. Loss of receptor on tuberculin-reactive T-cells marks active pulmonary tuberculosis. PLoS ONE. 2:e735.
38. Trattevin, P. E. Casalino, L. Fleury, G. Egmann, M. Ruel, and E. Bouvet 1999. The validity of medical history, classic symptoms, and chest radiographs in predicting pulmonary tuberculosis: derivation of a pulmonary tuberculosis prediction model. Chest 115:1248-1253.
39. WHO 2005. Global tuberculosis control. Surveillance, planning, financing. WHO report, Geneva, Switzerland, 1-247.
40. Zhang, H., J. Wang, J. Lei, M. Zhang, Y. Yang, Y. Chen, and H. Wang, 2007. PPE protein (Rv3425) from DNA segment RD11 of Mycobacterium tuberculosis: a potential B-cell antigen used for serological diagnosis to distinguish vaccinated controls from tuberculosis patients. Clin. Microbiol. Infect. 13:139-145.
41. Kyte, J. and Doolittle, R. F., J. Mol. Biol. 157:105-132 (1982).
42. Jameson, B. A. and Wolf, H., Bioinformatics 4:181-186 (1988).
43. Chou, P. Y, and Fasman, G. D. Biochemistry 13:222-245 (1974).
44. Li, L., Munir, S., Bannantine, J., Sreevatsan, Kanijilal, S. and Kapur, V., “Rapid Expression of Mycobacterium avium subsp. Paratuberculosis Recombinant Proteins for Antigen Discovery, Clinical and Vaccine Immunology. 14:102-105 (2007).
45. Devi, K. R., K. S. Kumar, B. Ramalingam, and Alamelu R. 2002. Purification and characterization of three immunodominant proteins (38, 30, and 16 kDa) of Mycobacterium tuberculosis. Protein Expr. Purif. 24: 188-195.
46. Ljungqvist, L., A. B. Andersen, P. Andersen, K. Hasløv, A. Worsaae, J Bennedsen and I. Heron. 1990. Affinity purification, biological characterization and serological evaluation of defined antigens from Mycobacterium tuberculosis. Trop. Med. Parasitol. 41:333-335.
47. Zhang, H., J. Wang, J. Lei, M. Zhang, Y. Yang, Y. Chen, and H. Wang. 2007. PPE protein (Rv3425) from DNA segment RD11 of Mycobacterium tuberculosis: a potential B-cell antigen used for serological diagnosis to distinguish vaccinated controls from tuberculosis patients. Clin Microbiol Infect. 13:139-145.
48. Choudhary, R. K., S. Mukhopadhyay, P. Chakhaiyar, N. Sharma, K. J, Murthy, V. M. Katoch, and S. E. Hasnain. 2003, PPE1 antigen Rv2430c of Mycobacterium tuberculosis induces a strong B-cell response, Infect. Immun. 71:6338-6343.

### EQUIVALENTS

The present disclosure is not to be limited in terms of the particular embodiments described in this application. Many modifications and variations can be made without departing from its spirit and scope, as will be apparent to those skilled in the art. Functionally equivalent methods and apparatuses within the scope of the disclosure, in addition to those enumerated herein, will be apparent to those skilled in the art from the foregoing descriptions. Such modifications and variations are intended to fall within the scope of the appended claims. The present disclosure is to be limited only by the terms of the appended claims, along with the full scope of equivalents to which such claims are entitled. It is to be understood that this disclosure is not limited to particular methods, reagents, compounds compositions or biological systems, which can, of course, vary. It is also to be understood that the terminology used herein is for the purpose of describing particular embodiments only, and is not intended to be limiting.

In addition, where features or aspects of the disclosure are described in terms of Markush groups, those skilled in the art will recognize that the disclosure is also thereby described in terms of any individual member or subgroup of members of the Markush group.

As will be understood by one skilled in the art, for any and all purposes, particularly in terms of providing a written description, all ranges disclosed herein also encompass any and all possible subranges and combinations of subranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like include the number recited and refer to ranges which can be subsequently broken down into subranges as discussed above. Finally, as will be understood by one skilled in the art, a range includes each individual member. Thus, for example, a group having 1-3 cells refers to groups having 1, 2, or 3 cells. Similarly, a group having 1-5 cells refers to groups having 1, 2, 3, 4, or 5 cells, and so forth.

While various aspects and embodiments have been disclosed herein, other aspects and embodiments will be apparent to those skilled in the art. The various aspects and embodiments disclosed herein are for purposes of illustration and are not intended to be limiting, with the true scope and spirit being indicated by the following claims.

What is claimed is:

1. A method for detecting exposure to or diagnosing infection by Mycobacterium tuberculosis in a mammal comprising:
incubating a test biological sample from the mammal with an Rv1168c polypeptide comprising the amino acid sequence of SEQ ID NO: 1 or a fragment thereof, under conditions suitable for an antibody to bind to the Rv1168c polypeptide, or the fragment thereof, to form an Rv1168c polypeptide-antibody complex;
measuring the level of the Rv1168c polypeptide-antibody complex in the test biological sample; and
comparing the level of the Rv11668c polypeptide-antibody complex in the test biological sample to the level of Rv1168c polypeptide-antibody complex detected in a control sample,
wherein a greater level of the Rv1168c polypeptide-antibody complex in the test biological sample compared to the level of the Rv1168c polypeptide-antibody complex in the control sample is indicative of exposure to or infection by Mycobacterium tuberculosis in the mammal.

2. The method of claim 1, wherein the control sample comprises a biological sample from a mammal which has not been exposed to Mycobacterium tuberculosis or a mammal vaccinated with Bacille Calmette-Guérin.

3. The method of claim 1, wherein the Rv1168c polypeptide consists of the amino acid sequence of SEQ ID NO: 1.

4. The method of claim 1, wherein the Rv1168c polypeptide comprising the amino acid sequence of SEQ ID NO: 1 is fused to a heterologous polypeptide.

5. The method of claim 4, wherein the heterologous polypeptide is a poly-histidine tag.

6. The method of claim 1, wherein the test biological sample is selected from the group consisting of: whole blood; sputum; blood serum; plasma; saliva; cerebrospinal fluid; and urine.

7. The method of claim 6, wherein the test biological sample is blood serum.

8. The method of claim 1, wherein the level of Rv1168c polypeptide-antibody complex in the test biological sample and the control sample are measured using an assay format selected from the group consisting of: an enzyme immunoassay; an enzyme-linked immunosorbent assay; a radioimmunoassay; a rapid flow through assay; and a competitive assay.

9. The method of claim 8, wherein the level of Rv1168c polypeptide-antibody complex in the test biological sample and the control sample are measured using an enzyme immunoassay format.

10. The method of claim 1, wherein the mammal displays symptoms of Mycobacterium tuberculosis infection.

11. The method of claim 1, wherein the level of Rv1168c polypeptide-antibody complex detected in the control sample is a historical level from a reference sample.

12. The method of claim 1, wherein the Mycobacterium tuberculosis infection is an extrapulmonary Mycobacterium tuberculosis infection or the mammal's sputum is smear-negative for acid-fast bacilli.

13. A method for detecting active Mycobacterium tuberculosis infection in a mammal comprising:
contacting a test population of peripheral blood mononuclear cells (PBMCs) from the mammal with an Rv1168c polypeptide comprising the amino acid sequence of SEQ ID NO: 1 or a fragment thereof;
measuring the level of an at least one cytokine expressed by the test population of PBMCs; and
comparing the level of the at least one cytokine expressed by the test population of PBMCs to the level of the at least cytokine measured in a reference population of PBMCs,
wherein the expression of a greater level of the at least one cytokine in the test population of PBMCs compared to the level of the at least one cytokine in the reference population of PBMCs is indicative of active Mycobacterium tuberculosis infection in the mammal.

14. The method of claim 13, wherein the reference population of PBMCs is from a mammal not exposed to Mycobacterium tuberculosis or a mammal vaccinated with Bacille Calmette-Guérin.

15. The method of claim 13, wherein the level of the at least one cytokine expressed by the test population of PBMCs is at least about two times greater than the level of the at least one cytokine measured in the reference population of PBMCs.

16. The method of claim 13, wherein the at least one cytokine is a gamma interferon or an interleukin-5.

17. The method of claim 13 wherein the Rv1168c polypeptide consists of the amino acid sequence of SEQ ID NO: 1.

18. The method of claim 13, wherein the Rv1168c polypeptide is fused to a heterologous polypeptide.

19. The method of claim 18, wherein the heterologous polypeptide is a poly-histidine tag.

20. The method of 13, wherein the level of the at least one cytokine expressed by the test population of PBMCs is measured using an assay format selected from the group consisting of: an enzyme immunoassay; an enzyme-linked immunosorbent assay; a radioimmunoassay; and a two-site sandwich enzyme immunoassay.

21. The method of claim 20, wherein the level of the at least one cytokine expressed by the test population of PBMCs is measured using a two-site sandwich enzyme immunoassay.

22. The method of 13, wherein the mammal displays symptoms of Mycobacterium tuberculosis infection.

23. The method of 13 wherein the test population of PBMCs is incubated between about 1 day and about 6 days prior to measuring the level of the at least one cytokine expressed by the test population of PBMCs.

24. The method of claim 13, wherein the Mycobacterium tuberculosis infection is an extrapulmonary Mycobacterium tuberculosis infection or the mammal's sputum is smear-negative for acid-fast bacilli.

25. A method for detecting exposure to or diagnosing infection by Mycobacterium tuberculosis in a mammal comprising:
incubating a test biological sample from the mammal with an Rv1168c polypeptide binding agent under conditions suitable for the Rv1168c polypeptide binding agent to bind an Rv1168c polypeptide, or a fragment thereof, and form an Rv1168c polypeptide binding agent-Rv1168c polypeptide complex; and
determining the presence or absence of the Rv1168c polypeptide binding agent-Rv1168c polypeptide complex in the test biological sample,
wherein the presence of the Rv1168c polypeptide binding agent-Rv1168c polypeptide complex in the test biological sample is indicative of exposure to or infection by Mycobacterium tuberculosis in the mammal.

26. The method of claim 25, wherein the Rv1168c polypeptide binding agent is an anti-Rv1168c polypeptide antibody, or fragment thereof.

27. The method of claim 25, wherein the test biological sample is selected from the group consisting of whole blood; sputum; blood serum; plasma-saliva; cerebrospinal fluid; and urine.

28. The method of claim 27, wherein the test biological sample is blood serum.

29. A kit for assaying for anti-Rv1168c polypeptide antibody in a biological sample which comprises an Rv1168c polypeptide, or fragment thereof, and instructions for its use.

30. A kit for assaying for Rv168c polypeptide, or a fragment thereof in a biological sample which comprises an anti-Rv1168c polypeptide antibody, or fragment thereof, and instructions for its use.
